(19) United States
(12) Patent Application Publication
(10) Pub. No.: US 2014/0058184 A1
(43) Pub. Date: Feb. 27, 2014

(54) USE OF NEURAL STEM CELLS FOR TREATMENT OF MALIGNANCY

(71) Applicant: Susan E. Crawford, Chicago, IL (US)

(72) Inventor: Susan E. Crawford, Chicago, IL (US)

(21) Appl. No.: 13/593,643

(22) Filed: Aug. 24, 2012

(51) Int. Cl. A61K 35/30; A61N 5/10

(52) U.S. Cl. CPC A61K 35/30; A61N 5/10
USPC 600/1; 424/93.7; 604/522

(57) ABSTRACT

An improved method for treatment of malignancies is based on the interaction between nerve cells and angiogenesis of malignancies. In general, the method comprises: (1) harvesting neural stem cells; (2) culturing neural stem cells under conditions such that the cells proliferate while retaining their ability to differentiate; (3) applying a biocompatible adhesive to a post-surgical site of a malignancy; and (4) following the application of the biocompatible adhesive to the post-surgical site of the malignancy, applying the cultured neural stem cells to the post-surgical site of the malignancy to remove residual tumor cells and to induce endothelial cell apoptosis. The method can further comprise the administration of a therapeutically effective quantity of at least one anti-neoplastic therapeutic agent or administration of a therapeutically effective quantity of anti-neoplastic ionizing radiation. The invention further encompasses kits for use in treating malignancies.

### FIELD OF THE INVENTION

This application is directed to methods and compositions for use of neural stem cells for treatment of non-neural malignancies.

### BACKGROUND OF THE INVENTION

Although a number of new therapies have become available, cancer is still one of the most feared diseases. In 2007, cancer was estimated to have caused about 8 million deaths worldwide. Although a number of therapies for cancer exist and are applied in medical practice, nearly all of these therapies fall into three general categories: surgery, radiation, and chemotherapy. All of these therapies have risks and potential side effects, which can be serious. For example, surgery can lead to scarring and infection; it may also fail to remove all of the malignant tissue, leading to potential recurrence and even metastasis. Radiation can be accompanied by side effects such as stomatitis, nausea, diarrhea, fibrosis, lymphedema, proctitis, cystitis, cognitive disorders, and even causation of new malignancies. Chemotherapy can cause immunosuppression and myelosuppression, which can in turn cause susceptibility to opportunistic infections, bleeding, or anemia, as well as nausea, vomiting, cardiotoxicity, hepatotoxicity, nephrotoxicity, ototoxicity, or encephalopathy. Another aspect of cancer treatment is that many cancer treatments are highly specific as to the type of cancer against which they are effective, and, in some cases, are only effective in patients possessing particular genetic markers.

One potential unexplored area of cancer treatment is the possibility of exploiting interactions between nerve cells and vascularization to inhibit angiogenesis of tumors and thereby retard the growth of tumors and make them more susceptible to anti-cancer therapeutic agents.

In the embryo, nerves and endothelial cells migrate along parallel pathways and, in some instances, they are so intimately linked that when pathology strikes just one of these cell types, survival of both is in jeopardy (1). The crosstalk between these two cell populations during development has been extensively studied; however, communication between nerves and vessels in the tumor microenvironment is not as well understood. The histologic finding of perineural invasion by tumors is a significant risk factor for poor outcome in tumors of all types including pancreas (4), prostate (2), and the colorectal region (3). The mechanism underlying this heightened risk of progressive disease has been elusive. Unlike arteries, veins, and lymphatics, no lumen exists in nerves, thus, there is no direct conduit for distant dissemination of tumor cells.

Therefore, there exists a need for a method of cancer treatment that exploits the interaction between nerve cells and tumor proliferation. Such a method would provide a completely new route for treatment of malignancy, and could provide a route to treatment of many malignancies for which current anti-neoplastic medications, radiation treatments, and surgical procedures have proven substantially ineffective.

### SUMMARY OF THE INVENTION

The present invention provides a new route and mechanism for treatment of malignancies that is based on the previously unexploited interaction between neural stem cells and angiogenesis of tumor tissue. The use of this interaction provides a new avenue for treatment of a wide range of malignancies and meets the needs described above.

Methods according to the present invention can be used along with conventional methods of cancer therapy and contribute to their effectiveness. They are expected to be free of side effects and contribute to the retention of function in tissues and organs affected by malignancies by assisting in reversing denervation, a factor in pain and loss of function in many cases of malignancy.

One aspect of the invention is a method for treating a malignancy comprising the steps of:

(1) harvesting neural stem cells;

(2) culturing neural stem cells under conditions such that the cells proliferate while retaining their ability to differentiate;

(3) applying a biocompatible adhesive to a post-surgical site of a malignancy; and

(4) following the application of the biocompatible adhesive to the post-surgical site of the malignancy, applying the cultured neural stem cells to the post-surgical site of the malignancy to remove residual tumor cells and to induce endothelial cell apoptosis.

In one alternative, the step of harvesting neural stem cells is performed by harvesting neural stem cells from bone marrow. In another alternative, the step of harvesting neural stem cells is performed by harvesting neural stem cells from adipose tissue. In yet another alternative, the step of harvesting neural stem cells is performed by harvesting neural stem cells derived from hematopoietic stem cells. In still another alternative, the step of harvesting neural stem cells is performed by harvesting neural stem cells derived from Schwann cells from peripheral nerve biopsies.

Typically, the step of harvesting the neural stem cells is performed by using a high efficiency cell sorter to retrieve the neural stem cells. Typically, the high efficiency cell sorter sorts cells by fluorescence-activated cell sorting (FACS). Typically, the fluorescence-activated cell sorting employs fluorochrome-conjugated antibodies, and the fluorochrome-conjugated antibodies specifically bind a marker selected from the group consisting of nestin, PAX6, CD44, SOX2, RC2, BLBP, and GLAST. In another alternative, the step of harvesting the neural stem cells is performed by magnetic separation.

Typically, the harvested stem cells are cultured. Typically, proliferation of the harvested neural stem cells is stimulated by treatment with at least one growth factor selected from the group consisting of bFGF (basic fibroblast growth factor), EGF, TGF-α, and aFGF. Typically, the harvested stem cells are cultured in a culture medium that is DMEM/F12 (Invitrogen)) supplemented with N2 (Invitrogen), B27 (Invitrogen), 2 μg/ml heparin, 100 U/ml penicillin, and 100 μg/ml streptomycin. The culture medium can include at least one growth-promoting additive selected from the group consisting of insulin, transferrin, sodium selenite, putrescine, and progesterone; the culture medium can further include at least one additional growth-promoting factor selected from the group consisting of pituitary extract, antibiotics, and fetal calf serum.

In one preferred alternative, the neural stem cells are obtained from a subject to be treated, thus avoiding possible tissue mismatching and the resultant possibility of immune rejection.

In a preferred alternative, the biocompatible adhesive is a DOPA-substituted polyethylene glycol polymer. Typically, the DOPA-substituted polyethylene glycol polymer has the structure

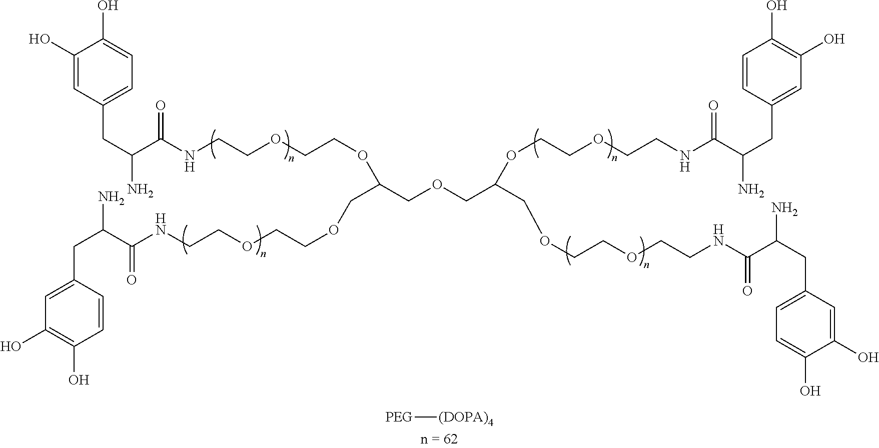

wherein n is from about 20 to about 100. Preferably, n is from about 40 to about 80. More preferably, n is 62.

In yet another alternative, the biocompatible adhesive is selected from the group consisting of fibrin glues and alkyl-α-cyanoacrylate glues.

Typically, the neural stem cells induce endothelial cell apoptosis. Typically, the neural stem cells release at least one anti-angiogenic protein. Typically, the at least one anti-angiogenic protein is selected from the group consisting of PEDF (pigment epithelium-derived factor), TSP (thrombospondin), and angiostatin.

In one alternative, the neural stem cells are applied to the post-operative site of the malignancy with at least one survival factor selected from the group consisting of pigment epithelium derived factor (PEDF) and propranolol.

The method can further comprise the step of administering a therapeutically effective quantity of an additional anti-neoplastic therapeutic agent.

The additional anti-neoplastic therapeutic agent can be selected from the group consisting of a nitrogen mustard, an alkylating agent, an alkyl sulfonate, a nitrosourea, a triazene, a folic acid analogue, a pyrimidine analogue antimetabolite, a purine analogue antimetabolite, a vinca alkaloid, a taxane, a camptothecin, an antibiotic, an enzyme, a biological response modifier, a platinum coordination complex, an anthracenedione, a substituted urea, a substituted hydrazine, an adrenocortical suppressant, a tyrosine kinase inhibitor, an adrenocorticosteroid, a progestin, an estrogen, an antiestrogen, an androgen, an antiandrogen, a gonadotropin-releasing hormone analogue, a monoclonal antibody, an interferon, an epidermal growth factor receptor (EGFR) kinase inhibitor, a vascular growth factor receptor (VGFR) kinase inhibitor, a fibroblast growth factor receptor (FGFR) kinase inhibitor, a platelet derived growth factor receptor (PDGF) kinase inhibitor, a Bcr-Ab1 kinase inhibitor, an antisense molecule, a non-steroidal anti-inflammatory drug, a topoisomerase inhibitor, a pro-apoptotic Bcl-2 family protein, a photodynamic compound, a radiodynamic compound, an immune suppressant, and an anti-angiogenic agent.

Specific additional anti-neoplastic therapeutic agents can include mechlorethamine, cyclophosphamide, ifosfamide, melphalan, chlorambucil, hexamethylmelamine, thiotepa, busulfan, carmustine, streptozocin, dacarbazine, temozolomide, methotrexate, 5-fluorouracil, cytarabine, gemcitabine, 6-mercaptopurine, 6-thioguanine, pentostatin, vincristine, paclitaxel, docetaxel, topotecan, dactinomycin, daunorubicin, doxorubicin, bleomycin, mitomycin C, L-asparaginase, interferon-alfa, interleukin-2, cisplatin, carboplatin, mitoxantrone, hydroxyurea, N-methylhydrazine, mitotane, aminoglutethimide, imatinib, prednisone, prednisolone, methylprednisolone, dexamethasone, betamethasone, triamcinolone, hydroxyprogesterone caproate, medroxyprogesterone acetate, megestrol acetate, diethylstilbestrol, ethinyl estradiol, tamoxifen, anastrozole, testosterone propionate, fluoxymesterone, flutamine, leuprolide, trastuzumab, rituximab, alemtuzumab, bevacizumab, cetuximab, gemtuzumab, ibritumomab, panitumumab, tositumomab, raloxifene, bicalutamide, finasteride, ketoconazole, fludarabine, Mylotarg, etoposide, staurosprine, altretamine, capecitabine, cladribine, idarubicin, vinorelbine, geranylgeraniol, lomustine, semustine, hydroxymethylmelamine, plicamycin, azathioprine, 2-chlorodeoxyadenosine, 5-fluorodeoxyuridine, and batimistat.

In another alternative, the method can further comprise administration of a therapeutically effective quantity of anti-neoplastic ionizing radiation. the anti-neoplastic ionizing radiation can be administered by a method selected from the group consisting of X-ray administration, proton beam administration, brachytherapy, and radioisotope therapy.

In one alternative, the anti-neoplastic ionizing radiation is administered by brachytherapy with a radioisotope selected from the group consisting of cesium-137, cobalt-60, indium-192, iodine-125, palladium-103, and ruthenium-106. In another alternative, the anti-neoplastic ionizing radiation is administered by radiotherapy with a radioisotope selected from the group consisting of iodine-131, lutetium-177, yttrium-90, strontium-89, and samarium-153. In either alternative, the radioisotope can be conjugated to a targeting moiety selected from the group consisting of a hormone, a receptor target, and a monoclonal antibody.

Another aspect of the invention is a kit for use in treating malignancies comprising:

(1) a culture of viable neural stem cells such that the neural stem cells can be transplanted to a post-surgical site of the malignancy; and

(2) a biocompatible adhesive adhering the neural stem cells to the post-surgical site of the malignancy.

The kit can further comprise, separately packaged, instructions for use. The kit can also further comprise, separately packaged, at least one survival factor for the neural stem cells. The kit can also further comprise, separately packaged, at least one anti-neoplastic therapeutic agent. The kit can also further comprise, separately packaged, at least one applicator to either apply the biocompatible adhesive or the neural stem cells to the post-surgical site. The kit can be adapted for treatment of prostate cancer, in which case the kit can further comprise, separately packaged, at least one anti-neoplastic therapeutic agent suitable for the treatment of prostate cancer.

### BRIEF DESCRIPTION OF THE DRAWINGS

These and other features, aspects, and advantages of the present invention will become better understood with reference to the following description, appended claims, and accompanying drawings where:

FIG. 1 shows that nerves are anti-angiogenic. Serum-free conditioned medium (CM) were collected from fetal rat and human Schwann cells (SC), minced murine sciatic nerve (N) and fetal atrocytes (W615HF). (a) Western blots were probed with anti PEDF, TSP-1 and angiostatin (AS) antibodies. Angiogenic activity was tested in proliferation (b) and migration (c) assays using umbilical vein and microvascular endothelial cells, respectively. In (b), nerve CM at 10 μg/ml significantly inhibited migration when tested alone and in the presence of VEGF (*P<0.015). In (c), W615HF CM suppressed VEGF-induced activity (*P<0.018) due to the presence of PEDF as neutralizing antibody increased migration over baseline (**P<0.005) and with VEGF (***P<0.03). Tissues were analyzed by H&E and/or immunostaining. (d,e) Peripheral nerves showed angiostatin (d) and PEDF (e) positivity throughout the nerve. Notch-1 positivity was most intense in the nerve sheath (f, arrow). (g) In pancreatic cancer, tumors (T) track along the nerve (N) sheath. (h,i) TSU-Pr1 bladder cancer tumors (T) adjacent to the sciatic nerve (SN) were small and circumscribed (h) as opposed to tumors away from the nerve which were large and infiltrative (i). (j) Nerves in the matrix of pancreatic carcinomas showed replacement of the nerve sheath by a fibrous band (FB). (k,l) Evaluation of pancreatic cancers showed perineural inflammation with vacuolation (arrow) of the nerve sheath (k) and angiostatin-stained sections showed increased vascularization (vessels, arrowheads) in the perineural region and multifocal loss of angiostatin expression (brown stain) in the nerve sheath as compared to normal nerve (d).

FIG. 2 shows that that neural cells inhibit tumor growth. Xenograft tumors of TSU-Pr1 or PC-3 cancer cells were treated with intratumoral injection of rat or human Schwann cells, neural stem cell or PBS control. Gross examination showed that Schwann cell treatment caused significant reduction in the size of the xenograft tumors (a) and also of tumor weight (b,c). By H&E staining (d,e), neural stem cell treated tumors (T) showed large areas of necrosis (n) compared to controls (d) (low power). (f) Notch-1 immunostaining of Schwann cell treated tumors show nerve cells (N) surrounded by a pseudocapsule (C) (high power). (g,h) Large proliferating capillary hemangiomas reveal no S100 positive nerves (g) vs. S100 positive nerves (brown staining) which were readily apparent in involuting hemangiomas (h).

FIG. 3 shows that NF1 plays a significant role in the anti-angiogenesis properties of nerves and that Immunostaining for PEDF (a) and Factor VIII-related antigen (b) were performed on neurofibroma and MPNST tissues. (c) Microvessel density was significantly higher in MPNST patients (*P<0.05). (d) Serum-free conditioned media (CM) was collected from human neurofibroma cells and MPNST cell line ST88 and tested in a microvascular endothelial cell migration assay +/− neutralizing antibodies against VEGF and PEDF. With neutralizing PEDF antibody, migration was significantly increased over CM alone in both neurofibroma (P=0.0045) and MPNST (P=0.0065) cells. (e,f) 12.5 day old NF1 deficient (NF^{-/-}) and age-matched wild type (NF^{+/+}) embryos were harvested. Compared to wild type, mouse embryonic fibroblast CM from NF1 deficient embryos had increased angiogenic activity in a migration assay (e) and increased tissue microvessel density (f) which were statistically significant (*P<0.05).

FIG. 4 shows that intact nerves are a barrier to tumorigenesis. (a) Schwann cells showed a dose-response to VEGF in a proliferation assay that diminishes at its highest dose. (b-f) A VEGF-containing hydron pellet was implanted next to either intact or a damaged sciatic nerve in an in vivo mouse model. H&E staining showed that the intact nerve had mild capillary growth in response to local VEGF (b), while the damaged nerve did not and resulted in dysregulated endothelial proliferation and development of hemangiomatous-like lesions (c). When vessels were quantified (d), while angiogenesis was increased in the presence of the VEGF pellet by the intact nerve over that of the PBS control (*P=0.002), when the nerve was damaged, angiogenesis was 2-fold higher than the intact nerve (**P<0.00014). (e,f) Immunostaining showed that the intact nerve sheath maintained notch-1 expression while the damaged nerve did not. (g) A model of tumor-nerve-vessel interactions during perineural invasion and cancer progression is presented. Tumor cells (T) are initially confined to the region of the neural sheath (NS). With a permissive microenvironment, this barrier is breached, and the tumor invades the nerve itself which is accompanied by intraneural vascularization and subsequent nerve damage or loss.

FIG. 5 shows the attachment of neurospheres to polydopamine-coated surgical sutures.

FIG. 6 depicts schematically one embodiment of a cancer treatment method according to the present invention, depicting surgical removal of a tumor, followed by application of a biocompatible adhesive and then anti-angiogenic neural stem cells in order to remove any residual tumor cells and induce endothelial apoptosis.

FIG. 7 depicts schematically the process of obtaining neural stem cells for use in a method according to the present invention, showing harvesting of stem cells from bone marrow or adipose tissue, retrieval of the stem cells using a high efficiency cell sorter, stimulation of the growth of the stem cells, and eventual application of the stem cells embedded in biocompatible adhesive.

FIG. 8 depicts the entire process schematically, combining the steps shown in FIG. 5 and those shown in FIG. 7.

FIG. 9 shows a model of tumor-nerve-vessel interactions during perineural invasion and cancer progression. Tumor cells (T) are initially confined to the region of the neural sheath (NS). With a permissive microenvironment, this barrier is breached, and the tumor invades the nerve itself which is accompanied by intraneural vascularization and subsequent nerve damage or loss.

FIG. 10 shows that neurospheres are visible on a polydopamine surface.

FIG. 11 shows an attached neurosphere (NS) cultured with pancreatic adenocarcinoma (Panc-1) cells showing the effect of the neurospheres in promoting regression of the Panc-1 cells.

FIG. 12 shows a correlation between the tendency of a hemangioma to grow or regress and the degree of innervation of the hemangioma. FIG. 11(A) shows an aggressive hemangioma, while FIG. 12(B) shows a regressing hemangioma, both stained with anti-neurofilament antibody.

FIG. 13 is a photomicrograph showing perineural invasion by a tumor.

FIG. 14 shows that Schwann cells, nerves, and neural stem cells secrete biologically active inhibitors of angiogenesis. In FIG. 14, the left panel shows Western blots indicating the secretion of pigment epithelium-derived factor (PEDF), thrombospondin-1 (TSP-1), and angiostatin (AS) in human Schwann cells (SC) and murine sciatic nerve (N). The right panel shows immunophotomicrographs showing PEDF in human nerve, PEDF in neural stem cells, and angiostatin in nerve.

FIG. 15 is a graph showing that the angiogenesis-inhibiting activity of nerve conditioned medium blocks vascular endothelial growth factor (VEGF)-induced angiogenesis. In FIG. 15, cell number×10³ is plotted against the concentration of nerve conditioned medium (Nerve CM) in μg/mL. The filled circles () are results with Nerve CM. The open circles (∘) are results with Nerve CM plus VEGF. Negative controls (BSA alone) and positive controls (BSA plus VEGF) are shown.

FIG. 16 shows the anti-angiogenic activity of PEDF in a corneal neovascularization assay. The left panel of FIG. 16 shows the results with bFGF alone; the right panel of FIG. 16 shows the results with bFGF and PEDF. PEDF clearly counteracts the angiogenic activity of bFGF minimizing revascularization.

FIG. 17 shows that PEDF concentration, as estimated by immunophotomicroscopy, decreases when normal prostatic tissue (left panel, FIG. 17) is compared with low grade cancer (center panel, FIG. 17) or high grade cancer (right panel, FIG. 17).

FIG. 18 shows that damage to sciatic nerve adjacent to prostate tumor cells promotes invasive tumor growth. The left side of FIG. 18 shows a tumor near the nerve, while the right side of FIG. 18 shows a tumor with a damaged nerve.

FIG. 19 shows that Schwann cells and neural stem cells suppress experimental prostate tumor growth in vivo. The top left portion of FIG. 19 shows the size of the prostate, including tumor therein, in controls (leftmost two examples) and tumors in contact with Schwann cells (rightmost two examples), indicating that the Schwann cells shrink the tumors. The bottom left portion of FIG. 19 shows photomicrographs with a control (left example) and neural stem cells (right example), indicating that contact with neural stem cells induces necrosis of tumor cells. The right portion of FIG. 19 is a graph that shows the effect of contact with neural stem cells on tumor weight, clearly showing a substantial decrease in tumor weight caused by contact with neural stem cells.

FIG. 20 shows the significant therapeutic effect of neural stem cell treatment on the intratumoral vasculature of prostate cancer xenografts. The left panel of FIG. 20 is a photomicrograph showing the intratumoral vasculature of a prostate cancer xenograft with no treatment, showing pathologic angiogenesis with a complex branching pattern. The right panel of FIG. 20 is a photomicrograph showing the intratumoral vasculature of a prostate cancer xenograft with treatment with neural stem cells, showing reduced angiogenesis and normal branching.

FIG. 21 shows a proposed model for neural-vascular crosstalk in the tumor microenvironment. The tumor growth process proceeds from early tumor invasion to nerve vascularization and eventually to nerve damage or loss. As nerve density decreases, the concentration of angiogenesis inhibitors normally produced by nervous tissue also decreases. The angiogenesis inhibitors include PEDF, TSP-1, and angiostatin. As nerve density decreases and tumor growth progresses, there is an increase in tumor volume and tumor microvessel density (MVD). In FIG. 21, “NS” designates the nerve sheath, “AI” designates the angiogenesis inhibitors, and “T” designates the tumor.

### DETAILED DESCRIPTION OF THE INVENTION

I have postulated that cells of neural lineage that innervate organs act as formidable barriers to tumor growth by secreting a potent cocktail of anti-angiogenic factors. Moreover, Schwann cells in the neural sheath participate in this barrier function by expressing high levels of Notch-1, a factor found to be important in maintaining the neural stem cell niche with PEDF (5-7), and in orchestrating embryonic vascularization (11).

In normal tissue, inhibitors of angiogenesis dominate over inducers to maintain vascular quiescence. Tumors can evade the activity of inhibitors through proteolytic degradation leading to a concurrent loss of neural cell survival factors. Normal nerves have a relative paucity of vessels; however, in diabetes, where VEGF is in excess, peripheral nerve vascularization is associated with degenerative changes in Schwann cells (12) and distal denervation (13) One interesting study of pancreatic cancer neural remodeling found a nearly 5 fold increase in nestin positive nerves (14). The reason for this shift to nestin positivity is unclear although it could represent a reactivation of early developmental pathways involving PEDF and Notch-1, known to be essential in renewal and maintenance of the neural stem cell population (5-7).

Pathological analysis of cancer over the years has led some to conclude that tumors actually lack innervation which is further supported by other studies that failed to find nerves in liver metastases (15). The absence of nerves made these authors conclude that nerves did not contribute to tumorigenesis, although another study linked paucity of nerves to angiogenesis when they assessed innervation of human xenograft tumors and found immunoreactive nerve fibers were, for the most part, absent in the areas of tumor angiogenesis (15). Changes in nerve density appear to influence the propensity for disease. In the prostate, an organ prone to disease with progressive age, nerve density diminishes with age, including within the peripheral zone where most prostate cancers occur (17). Even more intriguing is the unexpected observation in patients who underwent vagotomy for benign peptic ulcer disease where long term follow up showed a 1.3-3 fold increased risk of a wide spectrum of cancers. (18) Supporting this observation, in a murine breast cancer model, vagotomy increased the number of metastases (19). Here, evidence is provided to suggest that cells of neural lineage act as an organ's intrinsic anti-tumor agents by participating in a paracrine feedback loop to block angiogenesis while sustaining their own survival. These data suggest that while nerve-sparing surgical procedures are often performed to preserve function, they have the potential to retain an important natural anti-tumor barrier.

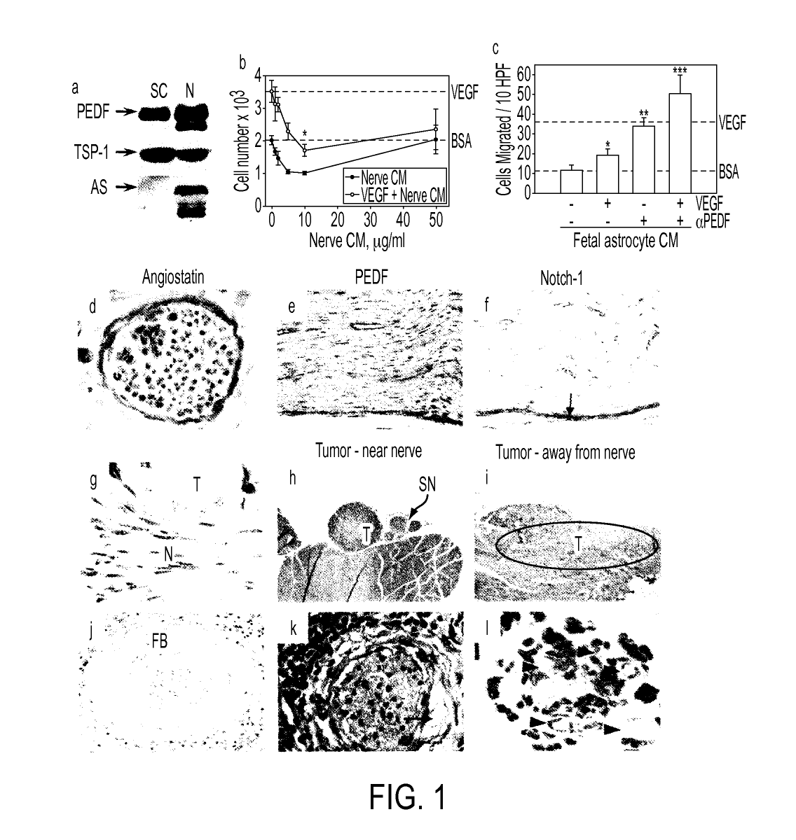

The tumor microenvironment is composed of many cell types, but relatively little is known about the contribution made by nerves (20,21). As an integral member of nearly every organ, with the possible exception of the highly vascularized placenta, it seems likely that they have a functional role in maintaining a disease-free state. We started with an inquiry to identify factors responsible for the relatively low vascular anatomic structure of the normal nerve where collateral circulation is important. Conditioned media were collected from rat and human Schwann cells, freshly harvested murine sciatic nerves and neural stem cells. By Western blot, we detected expression of three of the most potent anti-angiogenic inhibitors (8), PEDF, thrombospondin-1 (TSP-1), and angiostatin (FIG. 1a). In angiogenesis assays, anti-angiogenic activity effectively blocked VEGF-induced endothelial cell proliferation and migration (FIG. 1b,c). To localize these factors in tissue, murine tissue (n=15) and human tissue from skin (n=12), pancreas (n=10), and prostate (n=10) were immunostained for each of the inhibitors. All of the inhibitors strongly immunolocalized to the entire nerve including the sheath, the site of Schwann cells, and an example of angiostatin and PEDF staining are shown (FIG. 1d,e; data not shown). Since Notch-1 is important in Schwann cell biology (22), immunostains were performed and it was found to localize most intensely along the nerve sheath (FIG. 1f). The pattern of staining suggests two potential defense systems to maintain nerve survival. The first defense is in the sheath, itself, through expressing Notch-1 and PEDF which can protect neural cells from pro-apoptotic stimuli (23, 24), and the second defense is the interior component of anti-angiogenic factors. This two tier defense system may explain, in part, the characteristic tracking of most tumor cells along the neural sheath (FIG. 1g) and a smaller subset exhibiting transmural infiltration of the nerve.

Anti-angiogenic factors are soluble factors where distance is likely to create a concentration gradient. To determine whether or not tumors exhibit a different growth pattern based on proximity to a nerve, in this case, the sciatic nerve, tumor cells were injected adjacent to and away from the nerve. It was found that tumors adjacent to the nerve were small, encapsulated and had low vascularity whereas tumors placed away from the nerve were larger, highly vascular with infiltrative borders (compare FIG. 1 h to FIG. 1i). It was concluded that proximity of tumor cells to soluble nerve-derived anti-angiogenic mediators can influence net growth of the tumor which may provide one explanation for the unexpected favorable outcome of neuroblastomas located near posterior ganglia (25).

Unlike normal organs where the matrix is relatively quiescent, the tumor microenvironment often has neoplastic cells secreting metalloproteinases to degrade the matrix and activated stellate cells secreting collagens (26). The morphological changes in the nerves were studied within the active desmoplastic stroma of human pancreatic tumors (n=22) and it was found that collagen replaced the neural sheath, the site for expression of Notch-1 (FIG. 1j) and there was inflammatory cuffing of the nerves with tumor infiltration and evidence of intra-neural vascularization (FIG. 1k,l). These data stress the importance of the stroma as an active participant in nerve remodeling, and this gradual “takeover” of nerves may, in part, contribute to the unremitting pain associated with tumors.

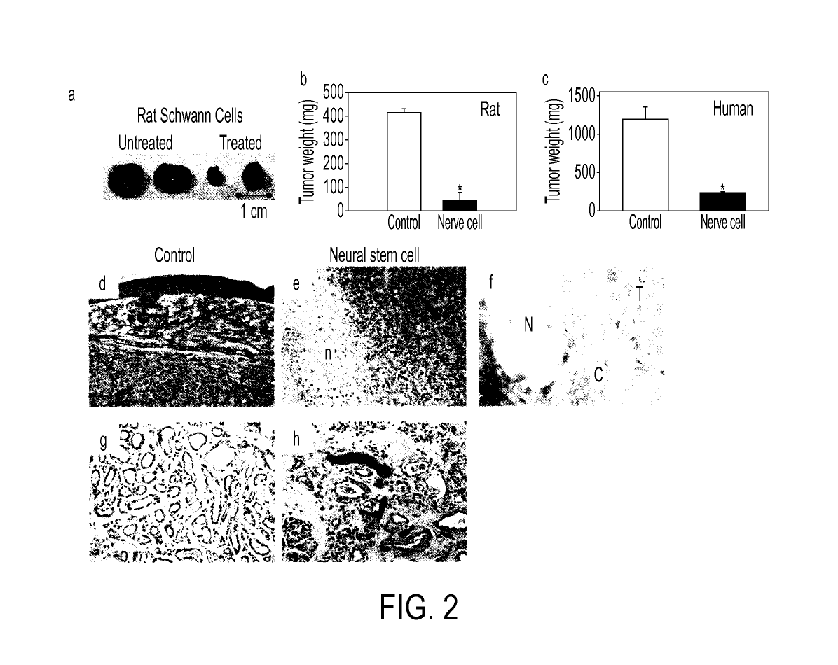

To explore the potential benefit of restoration of the neural cell population, the utility of Schwann cells and neural stem cells in vivo was tested Prostate cancer cells and bladder tumor cells were grown in xenograft models. When tumors reached 1 cm in diameter, Schwann cells, neural stem cells or vehicle alone was injected directly into the tumor and harvested the tumor two weeks later. When compared to the control group, the neural cell treated groups had significantly smaller tumor volumes with increased necrosis, less angiogenesis (FIG. 2a-e; data not shown), and were encapsulated by Notch-1 and PEDF expressing cells (FIG. 2f; data not shown). The data suggest that restoration of the neural network in the tumor microenvironment through Schwann cells or neural stem cells can effectively suppress tumor growth.

To better understand the in vivo interaction of nerves and vessels, hemangiomas, benign vascular tumors of childhood, were studied, since they have divergent biologic behavior with some requiring repeated resection while others undergo spontaneous involution. To determine if higher nerve density correlated with tumor regression and lower microvascular density, tissues were immunostained with neurofilament, S100, protein gene product 9.5 (PGP9.5), and Factor VIII-related antigen. Nerve density was nearly 3 fold higher in the involuted low vascular lesions (n=12) when compared to the proliferating high vascular hemangiomas (n=10; FIG. 2g,h; data not shown), suggesting that acquisition of nerves is an essential step in involution of hemangiomas and supporting a transcriptional profiling study demonstrating changes in neural markers (27).

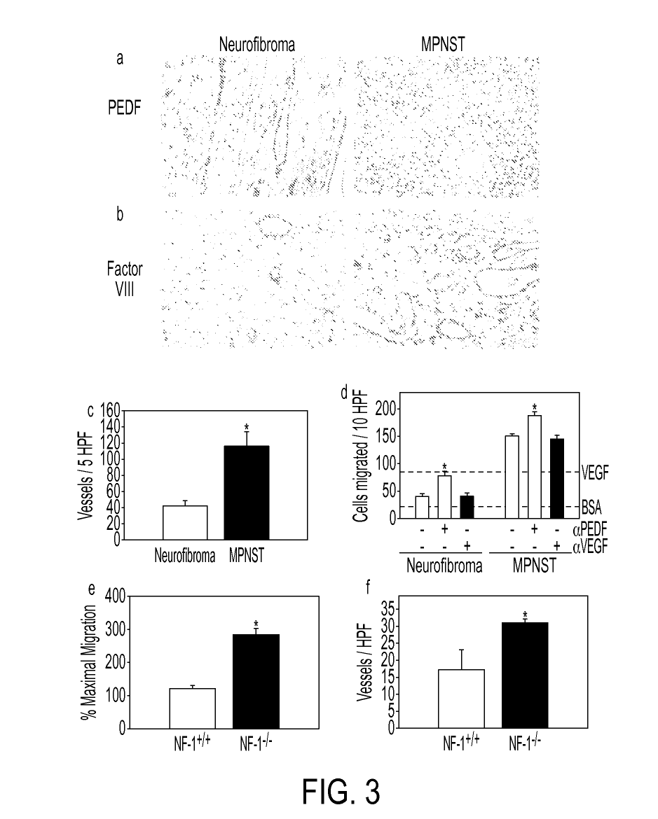

Not surprisingly, tumors of the peripheral nerve are uncommon, thus peripheral nerve lesions in neurofibromatosis type 1 (NF1) patients were evaluated. Benign neurofibromas (n=14) and malignant peripheral nerve sheath tumors (MPNST) (n=8) were immunostained for PEDF and Factor VIII-related antigen. The MPNST group had significantly higher microvessel density, less PEDF and breaks in the neural sheath unlike the PEDF-expressing low vascular benign neurofibromas (FIG. 3a-c). These data were supported by in vitro assays which showed that baseline angiogenic activity is higher in MPNST cells compared to neurofibroma cells, but in both, neutralizing PEDF function further increased activity (FIG. 3d). To assess whether altered NF1 correlated with overall angiogenic activity, 12.5 day old NF1 deficient and age-matched wild type embryos were harvested. NF1 deficient embryos had higher angiogenic activity and MVD (FIG. 3e,f). The observed angiogenic phenotype in NF1 deficiency appears to begin in development and a sequential loss of PEDF contributes to intraneural vascularization in MPNST.

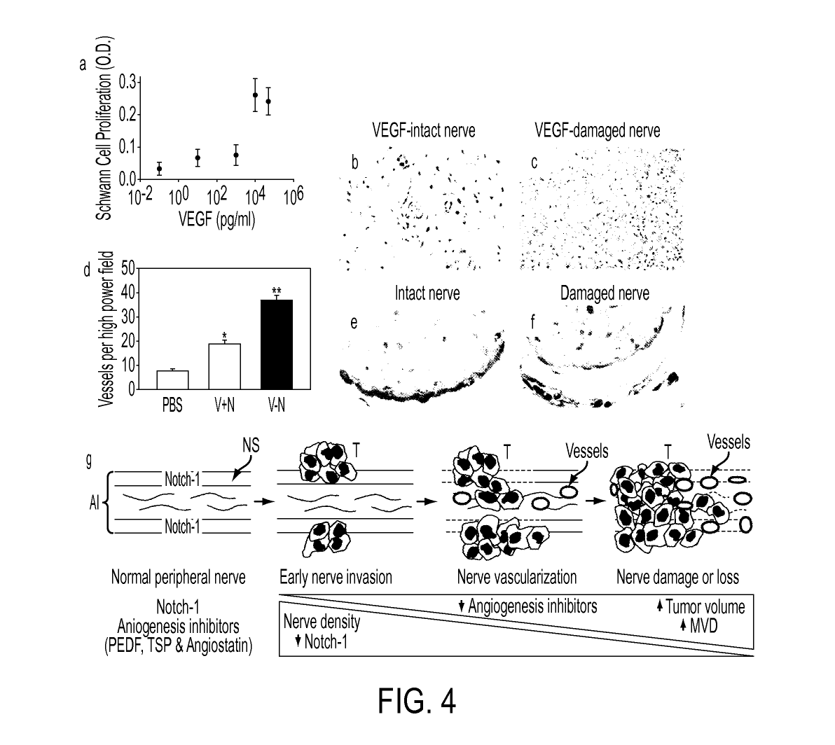

The experiments here have focused on anti-angiogenic factors and nerves; however, angiogenic inducer VEGF has similar actions on nerves by promoting survival and aiding repair of diabetes-induced neuropathy (28, 29). It was sought to determine if progressively higher levels of VEGF, simulating a tumor microenviroment, would continue to promote Schwann cell proliferation. It was found as levels of VEGF increased, Schwann cell proliferation reached a threshold and proliferative indices subsequently decreased (FIG. 4a). These data suggest that VEGF secretion would support Schwann cell growth in early tumorigenesis; however, this response would be dampened as the tumor enlarges and effaces an organ. To assess the relative contribution of the nerve-derived factors in a VEGF-rich environment, sciatic nerve sheaths were damaged or left intact. Interestingly, small hemangiomatous lesions developed when the VEGF angiogenic activity was un-opposed by factors arising from an intact nerve (FIG. 4b-d), and notch-1 expression had diminished (compare FIGS. 4e to 4f). This in vivo experiment suggests that loss of Notch-1 signaling in the neural sheath leads to unrestrained and aberrant VEGF-induced signaling to form atypical clusters of vessels or hemangiomatous lesions, not unlike a recent study showing an unexpected tumor response to pharmacologic blockade of Notch-1 (10).

From this study of nerve-tumor cell crosstalk, a perineural invasion model illustrating a pathway for a tumor-induced neuro-vascular switch is proposed (FIG. 4g). The normal peripheral nerve appears to have several lines of defense to assist in maintaining a tumor-free microenvironment. It secretes a wide spectrum of factors that have two important functions—blockade of blood vessel growth and maintenance of neural cell survival. As tumors advance into the microenvironment, they encounter the first line of defense for the nerve, the Notch-1 expressing neural sheath. In some instances, the tumors are held in check and continue to encroach only along the sheath, not quite able to overcome the pool of potent inhibitors of angiogenesis. In other instances, tumors are able to overcome the barrier, infiltrate the nerve and induce intra-neural vascularization, thereby creating access to the much needed lumina to disseminate to distant sites. The in vivo studies demonstrate that deficiencies in neural cell signaling within the tumor microenvironment can be effectively restored through a renewable source of anti-angiogenic factors, neural stem cells. Although nerve-sparing surgical procedures are performed to preserve function, this study highlights a previously unrecognized tumor suppressive benefit.

Methods

Cell and Tissue Cultures.

Serum-free conditioned medium was collected from fetal rat and human Schwann cells (kind gift from George DeVries, Loyola University of Chicago, Maywood, Ill.), fetal astrocytes (W615HF), murine neural stem cells, neurofibroma and MPNST (ST88) cells, murine sciatic nerve tissues, NF1 deficient and wild type embryonic fibroblast cultures. Neural stem cells were harvested as described (30).

Cryopreservation

Neurospheres on the second day of culture following passing were centrifuged at 1000 rpm for 5 min and resuspended in 7% DMSO and placed in cryogenic vials (final volume was 1.0 mL per vial). The vials were transferred to isopropanol freezing container and placed directly into an −80° C. freezer. The following day the cryogenic vials were dropped into liquid nitrogen for long-term storage.

Cryopreservation is described further in X.-H. Ma et al., “Slow-Freezing Cryopreservation of Neural Stem Cell Spheres with Different Diameters,” Cryobiology 60: 184-191 (2010), incorporated herein by this reference.

Cell Thawing

The cryogenic vials were taken out of liquid nitrogen as quickly as possible and put into thermostatic waterbath at 37° C. Quick agitation was conducted to increase the thawing rate. The cells were washed with culture medium, centrifuged at 1000 rpm for 5 min to remove the DMSO and resuspended and placed in culture medium (DMEM/F12 supplemented with N2, B27, and 20 ng/ml basic FGF).

Polydopamine Coating

Petri and tissue culture dishes (as well as coverslips) were flooded with polydopamine solution consisting of 10 mM dopamine hydrochloride (Sigma) dissolved in Tris Buffer (pH 8.5) and left overnight. The excess fluid was aspirated and the dishes were allowed to dry. These polydopamine coated dishes (as well as coverslips) were subsequently used for culture of neurosheres which attached to the surface and maintained viability.

Protein Expression

Proteins were resolved by SDS-PAGE and electroblotted to Hybond-C membrane (Amersham). Blots were probed with antibodies against TSP-1 (Thermo Fisher), PEDF (8) or angiostatin (R&D Systems) antibodies as described (9).

Preparation and Use of Surgical Sutures

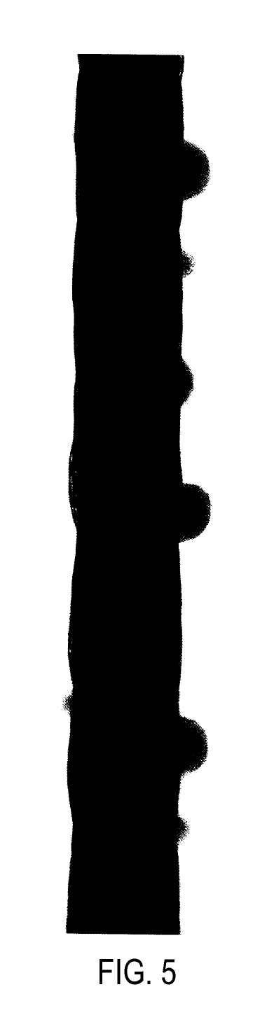

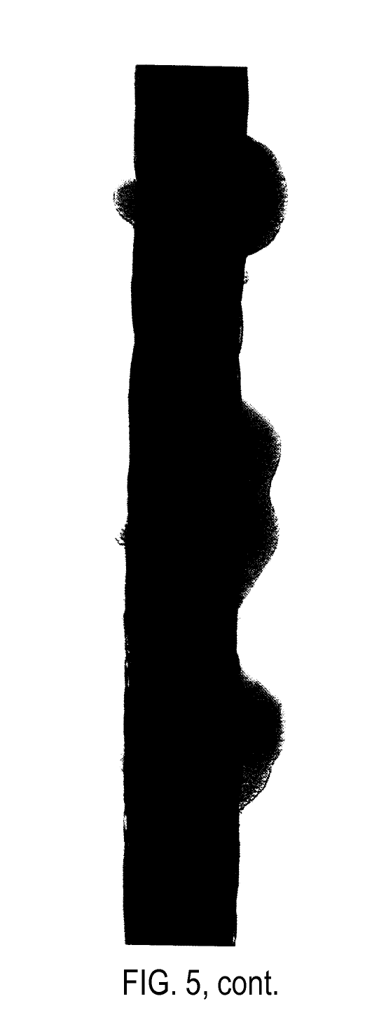

Nonadsorbable polyester “00” surgical suture was coated with the same polydopamine solution used for coating the culture dishes. The string was placed in as dish with cultured neurospheres on the third day following passage. Neurospheres attached to the string following overnight culture. Following transfer of the string to a new dish with neurosphere culture medium, the neurospheres attached to the string and were viable for over two weeks. The attachment of the neurospheres to the string is shown in FIG. 5 (two examples).

Isolation of Embryonic Neural Stem Cells and Establishment of Basic FGF-Generated Neurospheres

For isolation of embryonic neural stem cells and establishment of basic FGF-generated neurospheres, timed pregnant C57BL mice were sacrificed by cervical dislocation on day 13.5 of pregnancy. The embryos were removed and the lateral and median ganglionic eminence regions from each embryo were carefully dissected and dissociated by incubation in calcium- and magnesium-free PBS for 5 minutes, followed by mechanical dissociation. After a wash, the cells were resuspended in culture medium consisting of DMEM/F12 (Invitrogen)) supplemented with N2 (Invitrogen), B27 (Invitrogen), 2 μg/ml heparin (Sigma), 100 U/ml penicillin, and 100 μg/ml streptomycin. Basic FGF (Millipore) was then added to the medium at the final concentration of 20 ng/ml. Cells were cultured at 37° C. and 5% CO2 in 10 cm Petri dishes for 5 to 7 days and allowed to form neurospheres before the first passage. Neurospheres were subsequently passed every 3 to 4 days following dissociation into single cells with Accutase (PAA Laboratories) and cultured in the same conditions. This procedure is based on the procedure described in N. Israsena et al., “The Presence of FGF2-Signaling Determines Whether β-Catenin Exerts Effects on Proliferation or Neuronal Differentiation of Neural Stem Cells,” Dev. Biol. 268: 220-231 (2004), incorporated herein by this reference. Another method for generation of neurospheres employing Accutase® (StemPro®), a proteolytic enzyme of marine origin available from Life Technologies, Carlsbad, Calif., is described in H. Ahlenius & Z. Kokaia, “Isolation and Generation of Neurosphere Cultures from Embryonic and Adult Mouse Brain,” Methods Mol. Biol. 633: 241-252 (2010), incorporated herein by this reference. The components of N2 and B27 are described in F. P. Wachs et al., “High Efficacy of Clonal Growth and Expansion of Adult Neural Stem Cells,” Lab. Invest. 83: 949-962 (2003), incorporated herein by this reference. Specifically, N2 includes bovine serum albumin (BSA), transferrin, insulin, progesterone, putrescine, and sodium selenite. B27 includes BSA, progesterone, putrescine, sodium selenite, biotin, L-carnitine, corticosterone, ethanolamine, D(+)-galactose, glutathione (reduced), linolenic acid, linoleic acid, retinyl acetate, selenium, T3 (triodo-1-thyronine), DL-α-tocopherol (vitamin E), DL-α-tocopherol acetate, catalase, and superoxide dismutase.

In Vitro Assays.

Human microvascular and umbilical vein endothelial cells were used for angiogenesis assays. Migrations were performed as previously described (9), and proliferation assays were by MTT assay (Invitrogen). Positive (VEGF) and negative (BSA) controls and neutralizing antibodies were used as previously described (9).

In Vivo Experiments.

For xenografts, PC-3 (prostate) and TSU-Pr1 (bladder) cancer cells (2.5−5×10⁶) were injected into the right flank of nude mice. Starting when tumors reached ˜1 cm in diameter (˜day 13-14 post-cell injection), Schwann cells, neural stem cells or PBS was injected into the tumor every 2-3 days (3× weekly) and harvested two weeks later. For sciatic nerve proximity studies, minced xenograft tumor tissue (˜1-2 mm, TSU-Pr1) was placed either adjacent to or away from the nerve or a VEGF-containing hydron pellet (40 ng/pellet) was placed next to a nicked (injured) or sham-operated nerve.

Mouse and Human Tissue Analysis.

Mouse and human tissues were H&E stained and/or immunostained as described⁹ with antibodies against PEDF, TSP-1, angiostatin (angiogenesis inhibitors) notch-1 (neural survival factor), Factor VIII-related antigen (vessels), PGP9.5, neurofilament and S100 (nerves). The number of vessels was counted per high power field by a pathologist.

Statistics.

Groups were compared by a Student's t test with P<0.05 considered significant.

FIG. 1 shows that nerves are anti-angiogenic. Serum-free conditioned medium (CM) were collected from fetal rat and human Schwann cells (SC), minced murine sciatic nerve (N) and fetal atrocytes (W615HF). (a) Western blots were probed with anti PEDF, TSP-1 and angiostatin (AS) antibodies. Angiogenic activity was tested in proliferation (b) and migration (c) assays using umbilical vein and microvascular endothelial cells, respectively. In (b), nerve CM at 10 μg/ml significantly inhibited migration when tested alone and in the presence of VEGF (*P<0.015). In (c), W615HF CM suppressed VEGF-induced activity (*P<0.018) due to the presence of PEDF as neutralizing antibody increased migration over baseline (**P<0.005) and with VEGF (***P<0.03). Tissues were analyzed by H&E and/or immunostaining. (d,e) Peripheral nerves showed angiostatin (d) and PEDF (e) positivity throughout the nerve. Notch-1 positivity was most intense in the nerve sheath (f, arrow). (g) In pancreatic cancer, tumors (T) track along the nerve (N) sheath. (h,i) TSU-Pr1 bladder cancer tumors (T) adjacent to the sciatic nerve (SN) were small and circumscribed (h) as opposed to tumors away from the nerve which were large and infiltrative (i). (j) Nerves in the matrix of pancreatic carcinomas showed replacement of the nerve sheath by a fibrous band (FB). (k,l) Evaluation of pancreatic cancers showed perineural inflammation with vacuolation (arrow) of the nerve sheath (k) and angiostatin-stained sections showed increased vascularization (vessels, arrowheads) in the perineural region and multifocal loss of angiostatin expression (brown stain) in the nerve sheath as compared to normal nerve (d).

FIG. 2 shows that neural cells inhibit tumor growth. Xenograft tumors of TSU-Pr1 or PC-3 cancer cells were treated with intratumoral injection of rat or human Schwann cells, neural stem cell or PBS control. Gross examination showed that Schwann cell treatment caused significant reduction in the size of the xenograft tumors (a) and also of tumor weight (b,c). By H&E staining (d,e), neural stem cell treated tumors (T) showed large areas of necrosis (n) compared to controls (d) (low power). (f) Notch-1 immunostaining of Schwann cell treated tumors show nerve cells (N) surrounded by a pseudocapsule (C) (high power). (g,h) Large proliferating capillary hemangiomas reveal no S100 positive nerves (g) vs. S100 positive nerves (brown staining) which were readily apparent in involuting hemangiomas (h).

FIG. 3 shows that NF1 plays a significant role in the anti-angiogenesis properties of nerves and that Immunostaining for PEDF (a) and Factor VIII-related antigen (b) were performed on neurofibroma and MPNST tissues. (c) Microvessel density was significantly higher in MPNST patients (*P<0.05). (d) Serum-free conditioned media (CM) was collected from human neurofibroma cells and MPNST cell line ST88 and tested in a microvascular endothelial cell migration assay +/− neutralizing antibodies against VEGF and PEDF. With neutralizing PEDF antibody, migration was significantly increased over CM alone in both neurofibroma (P=0.0045) and MPNST (P=0.0065) cells. (e,f) 12.5 day old NF1 deficient (NF^{-/-}) and age-matched wild type (NF^{+/+}) embryos were harvested. Compared to wild type, mouse embryonic fibroblast CM from NF1 deficient embryos had increased angiogenic activity in a migration assay (e) and increased tissue microvessel density (f) which were statistically significant (*P<0.05).

FIG. 4 shows that intact nerves are a barrier to tumorigenesis. (a)

Schwann cells showed a dose-response to VEGF in a proliferation assay that diminishes at its highest dose. (b-f) A VEGF-containing hydron pellet was implanted next to either intact or a damaged sciatic nerve in an in vivo mouse model. H&E staining showed that the intact nerve had mild capillary growth in response to local VEGF (b), while the damaged nerve did not and resulted in dysregulated endothelial proliferation and development of hemangiomatous-like lesions (c). When vessels were quantified (d), while angiogenesis was increased in the presence of the VEGF pellet by the intact nerve over that of the PBS control (*P=0.002), when the nerve was damaged, angiogenesis was 2-fold higher than the intact nerve (**P<0.00014). (e,f)

Immunostaining showed that the intact nerve sheath maintained notch-1 expression while the damaged nerve did not. (g) A model of tumor-nerve-vessel interactions during perineural invasion and cancer progression is presented. Tumor cells (T) are initially confined to the region of the neural sheath (NS). With a permissive microenvironment, this barrier is breached, and the tumor invades the nerve itself which is accompanied by intraneural vascularization and subsequent nerve damage or loss.

FIG. 5 shows attachment of neurospheres to polydopamine-coated surgical sutures.

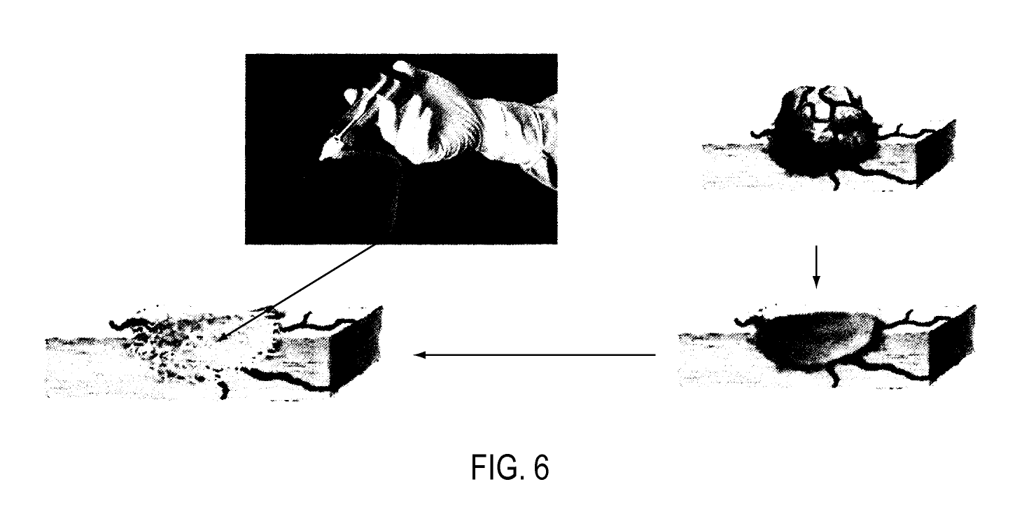

FIG. 6 depicts schematically one embodiment of a cancer treatment method according to the present invention, depicting surgical removal of a tumor, followed by application of a biocompatible adhesive and then anti-angiogenic neural stem cells in order to remove any residual tumor cells and induce endothelial apoptosis.

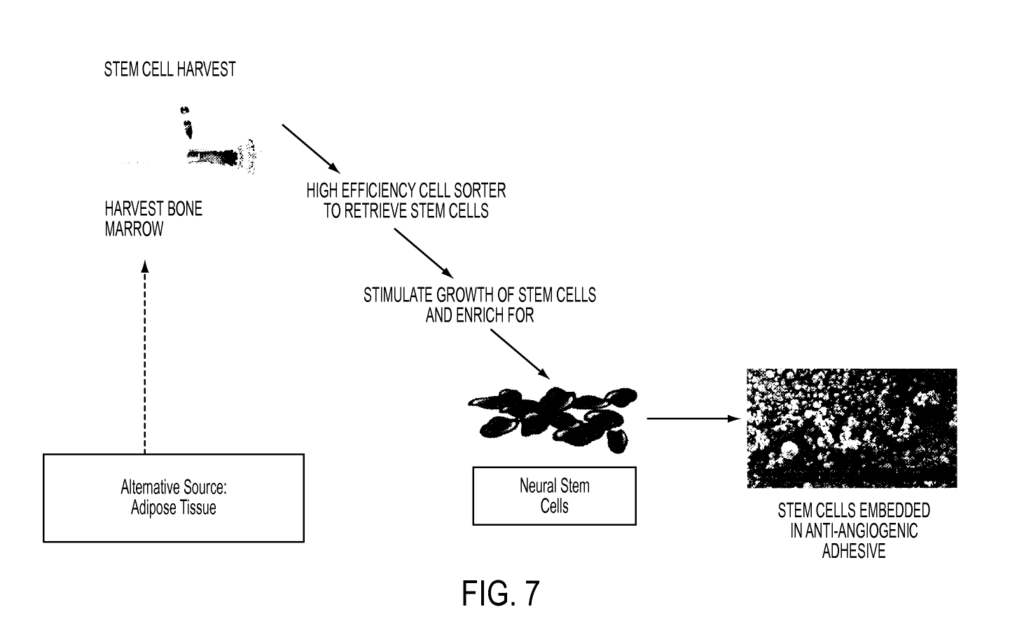

FIG. 7 depicts schematically the process of obtaining neural stem cells for use in a method according to the present invention, showing harvesting of stem cells from bone marrow or adipose tissue, retrieval of the stem cells using a high efficiency cell sorter, stimulation of the growth of the stem cells, and eventual application of the stem cells embedded in biocompatible adhesive.

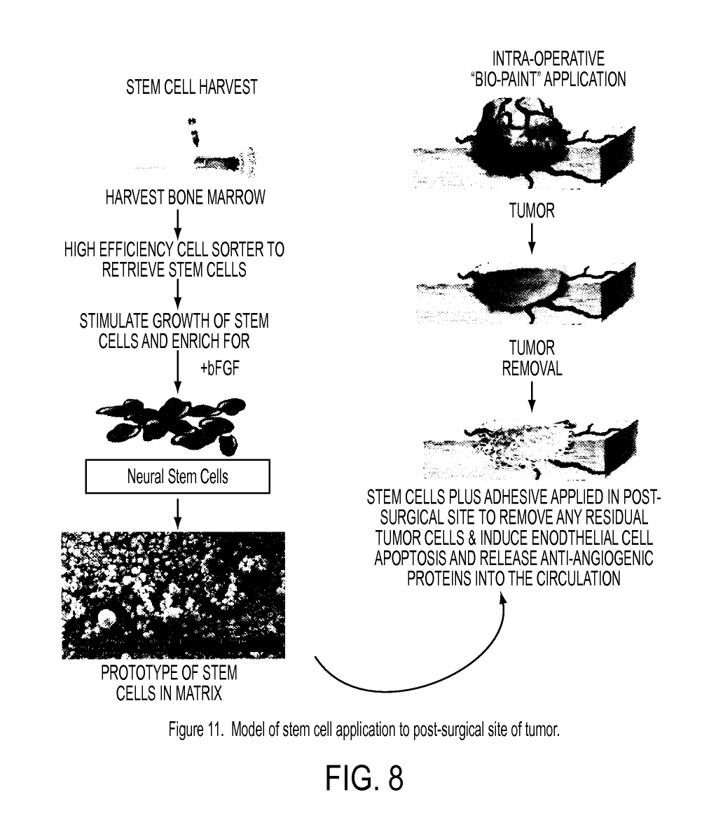

FIG. 8 depicts the entire process schematically, combining the steps shown in FIG. 6 and those shown in FIG. 7.

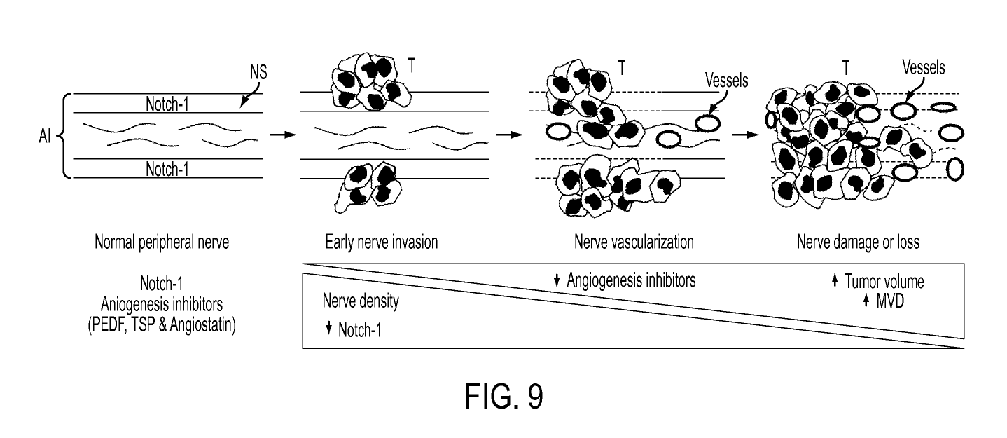

FIG. 9 shows a model of tumor-nerve-vessel interactions during perineural invasion and cancer progression is presented. Tumor cells (T) are initially confined to the region of the neural sheath (NS). With a permissive microenvironment, this barrier is breached, and the tumor invades the nerve itself which is accompanied by intraneural vascularization and subsequent nerve damage or loss.

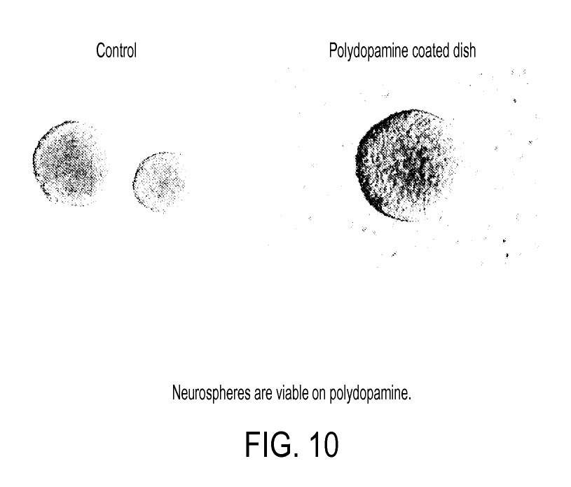

FIG. 10 shows that neurospheres are visible on a polydopamine surface.

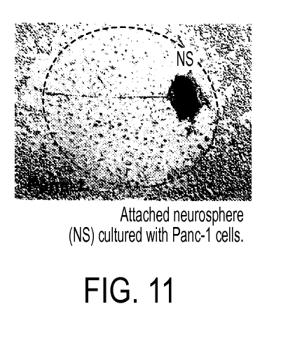

FIG. 11 shows an attached neurosphere (NS) cultured with pancreatic adenocarcinoma (Panc-1) cells showing the effect of the neurospheres in promoting regression of the Panc-1 cells.

In addition, I have demonstrated that neural stem cells can suppress prostate cancer growth by blocking pathogenic angiogenesis in vivo.

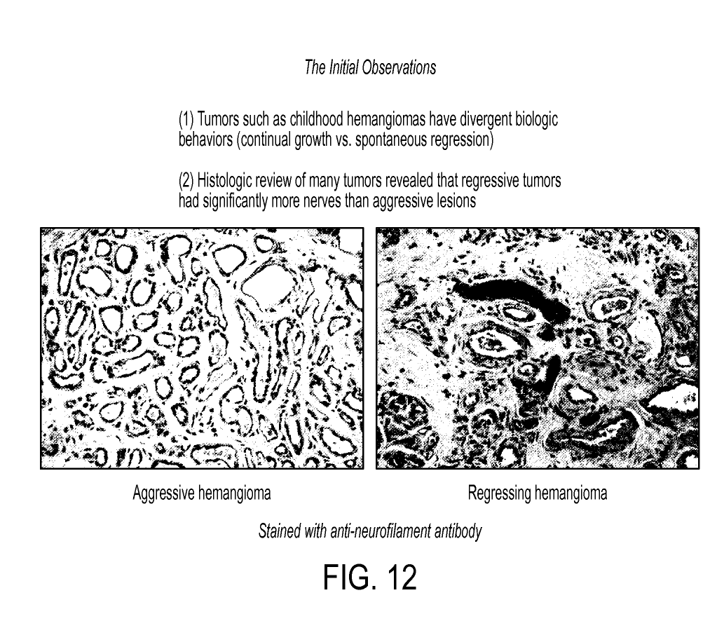

Tumors such as childhood hemangiomas have divergent biologic behaviors (continual growth versus spontaneous regression). Histologic review of many tumors revealed that regressive tumors had significantly more nerves than aggressive lesions. This is shown in FIG. 12. FIG. 12(A) shows an aggressive hemangioma, while FIG. 12(B) shows a regressing hemangioma, both stained with anti-neurofilament antibody.

This observation raises the question of whether nerves actively participate in making the microenvironment suppressive to tumors. The answer to this question can provide a novel mechanism for treatment of malignancies.

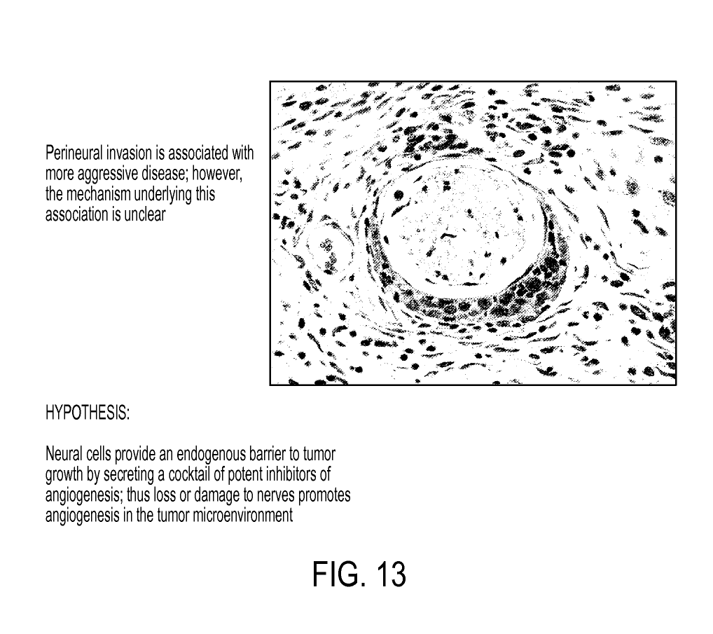

Perineural invasion by the tumor is associated with more aggressive disease; however, the mechanism of this is unclear. FIG. 13 is a photomicrograph showing perineural invasion. This leads to the hypothesis that neural cells provide an endogenous barrier to tumor growth by secreting a cocktail of potent inhibitors of angiogenesis; thus loss or damage to nerves promotes angiogenesis in the tumor microenvironment.

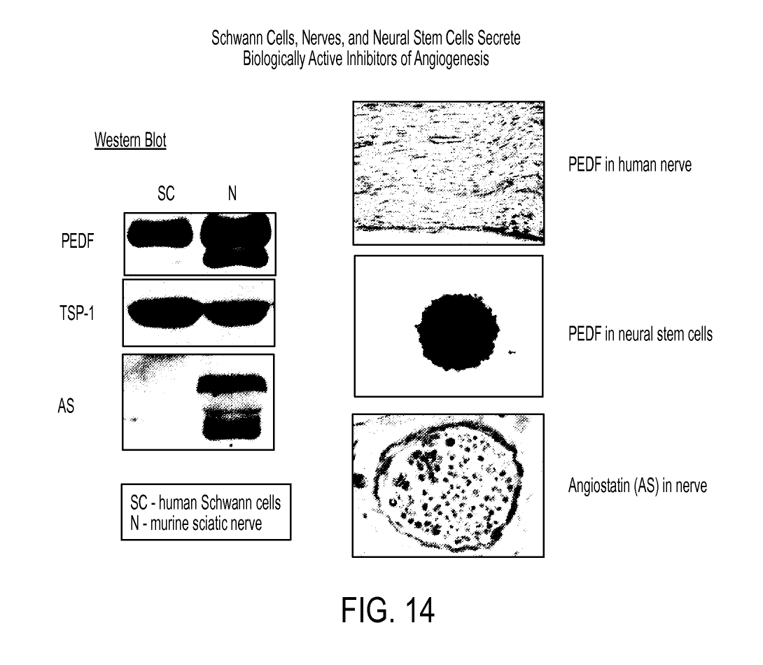

Schwann cells, nerves, and neural stem cells secrete biologically active inhibitors of angiogenesis. In FIG. 14, the left panel shows Western blots indicating the secretion of pigment epithelium-derived factor (PEDF), thrombospondin-1 (TSP-1), and angiostatin (AS) in human Schwann cells (SC) and murine sciatic nerve (N). The right panel shows immunophotomicrographs showing PEDF in human nerve, PEDF in neural stem cells, and angiostatin in nerve.

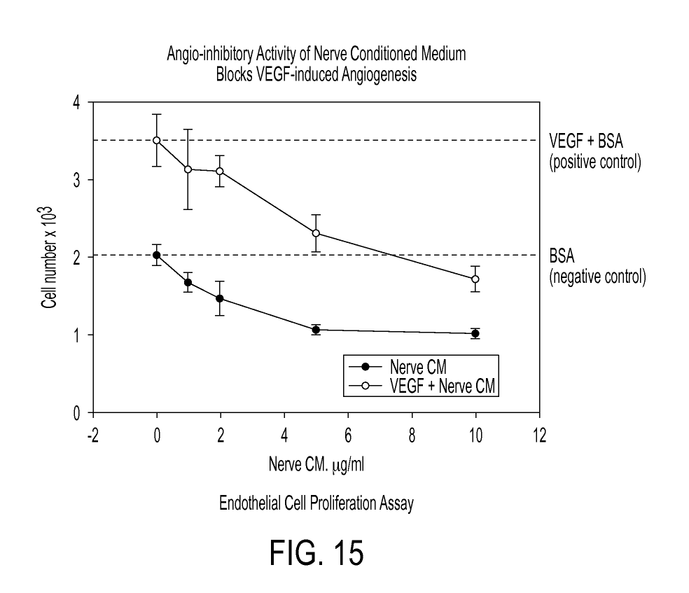

FIG. 15 is a graph showing that the angiogenesis-inhibiting activity of nerve conditioned medium blocks vascular endothelial growth factor (VEGF)-induced angiogenesis. In FIG. 15, cell number×10³ is plotted against the concentration of nerve conditioned medium (Nerve CM) in μg/mL. The filled circles () are results with Nerve CM. The open circles (∘) are results with Nerve CM plus VEGF. Negative controls (BSA alone) and positive controls (BSA plus VEGF) are shown.

Pigment epithelium-derived factor (PEDF) was initially isolated from retinal pigmented epithelium (J. Tombran-Tink et al., “PEDF: A Pigment Epithelium-Derived Factor with Potent Neuronal Differentiative Activity,” Exp. Eye Res. 53: 411-414 (1991), incorporated herein by this reference). PEDF is a 50 kDa secreted glycoprotein that circulates at high levels in the blood and is expressed by most normal epithelial and stromal tissues. PEDF is a potent inhibitor of angiogenesis (D. W. Dawson et al., “Pigment Epithelium-Derived Factor: A Potent Inhibitor of Angiogenesis,” Science 285: 245-248 (1999), incorporated herein by this reference).

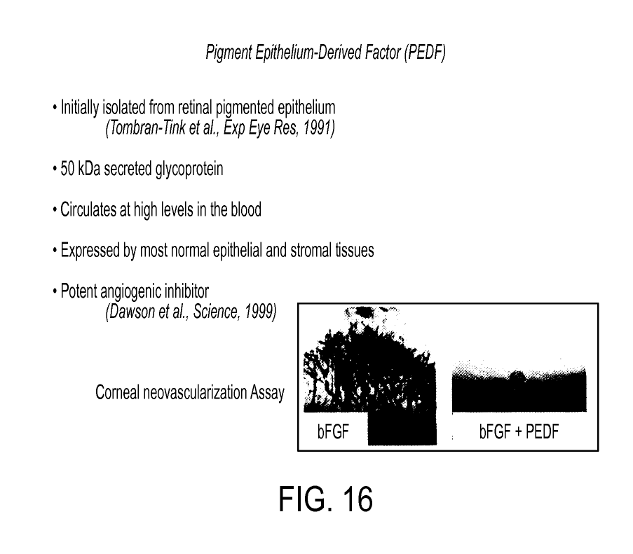

FIG. 16 shows the anti-angiogenic activity of PEDF in a corneal neovascularization assay. The left panel of FIG. 16 shows the results with bFGF alone; the right panel of FIG. 16 shows the results with bFGF and PEDF. PEDF clearly counteracts the angiogenic activity of bFGF minimizing revascularization.

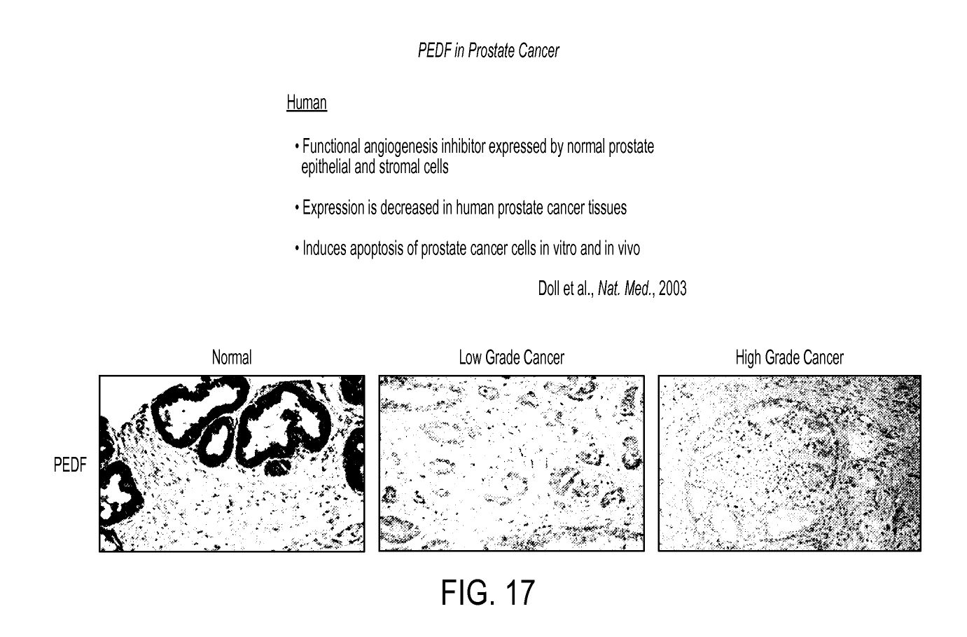

In prostate cancer, PEDF is clearly involved. PEDF, a functional angiogenesis inhibitor, is expressed by normal prostatic epithelial and stromal cells. The expression of PEDF is decreased in human prostate cancer tissues. PEDF also induces apoptosis of prostate cancer cells in vitro and in vivo (J. A. Doll et al., “Pigment Epithelium-Derived Factor Regulates the Vasculature and Mass of the Prostate and Pancreas,” Nature Med. 9: 774-780 (2003), incorporated herein by this reference). FIG. 17 shows that PEDF concentration, as estimated by immunophotomicroscopy, decreases when normal prostatic tissue (left panel, FIG. 17) is compared with low grade cancer (center panel, FIG. 17) or high grade cancer (right panel, FIG. 17).

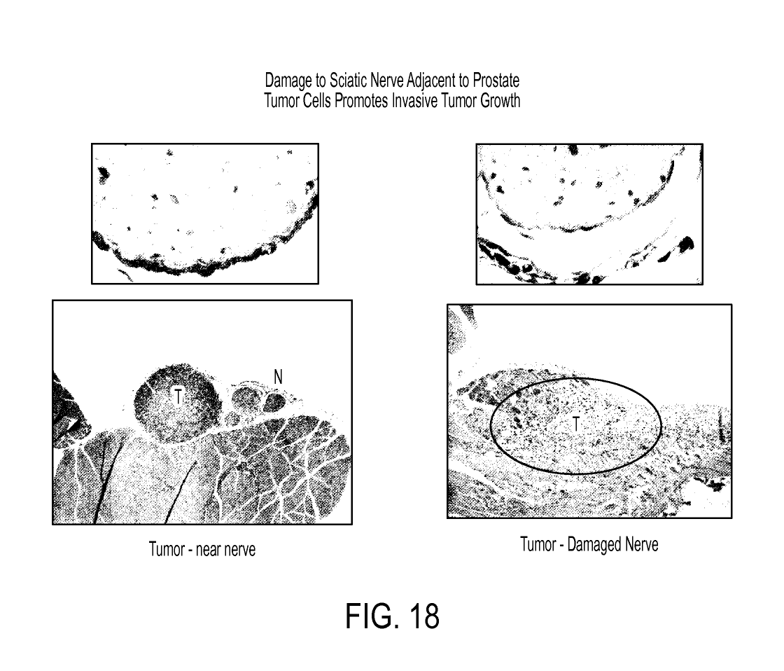

Damage to sciatic nerve adjacent to prostate tumor cells promotes invasive tumor growth, as shown in FIG. 18. The left side of FIG. 18 shows a tumor near the nerve, while the right side of FIG. 18 shows a tumor with a damaged nerve.

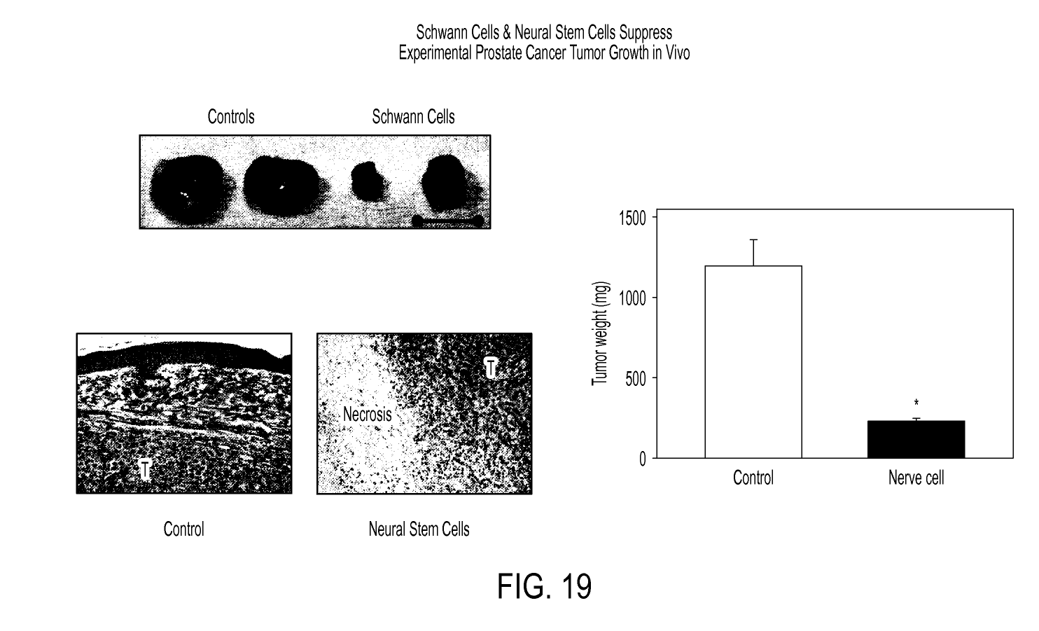

Schwann cells and neural stem cells suppress experimental prostate tumor growth in vivo. This is shown in FIG. 19. The top left portion of FIG. 19 shows the size of the prostate, including tumor therein, in controls (leftmost two examples) and tumors in contact with Schwann cells (rightmost two examples), indicating that the Schwann cells shrink the tumors. The bottom left portion of FIG. 19 shows photomicrographs with a control (left example) and neural stem cells (right example), indicating that contact with neural stem cells induces necrosis of tumor cells. The right portion of FIG. 19 is a graph that shows the effect of contact with neural stem cells on tumor weight, clearly showing a substantial decrease in tumor weight caused by contact with neural stem cells.

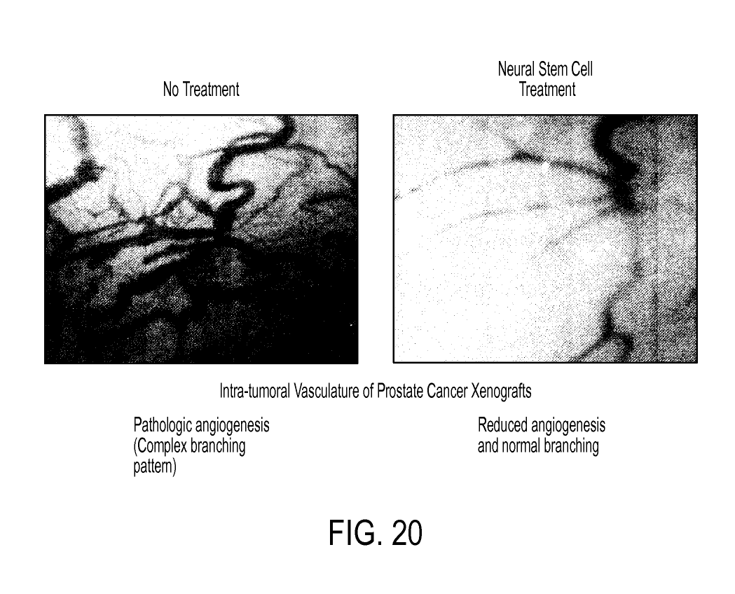

FIG. 20 shows the significant therapeutic effect of neural stem cell treatment on the intratumoral vasculature of prostate cancer xenografts. The left panel of FIG. 20 is a photomicrograph showing the intratumoral vasculature of a prostate cancer xenograft with no treatment, showing pathologic angiogenesis with a complex branching pattern. The right panel of FIG. 20 is a photomicrograph showing the intratumoral vasculature of a prostate cancer xenograft with treatment with neural stem cells, showing reduced angiogenesis and normal branching.

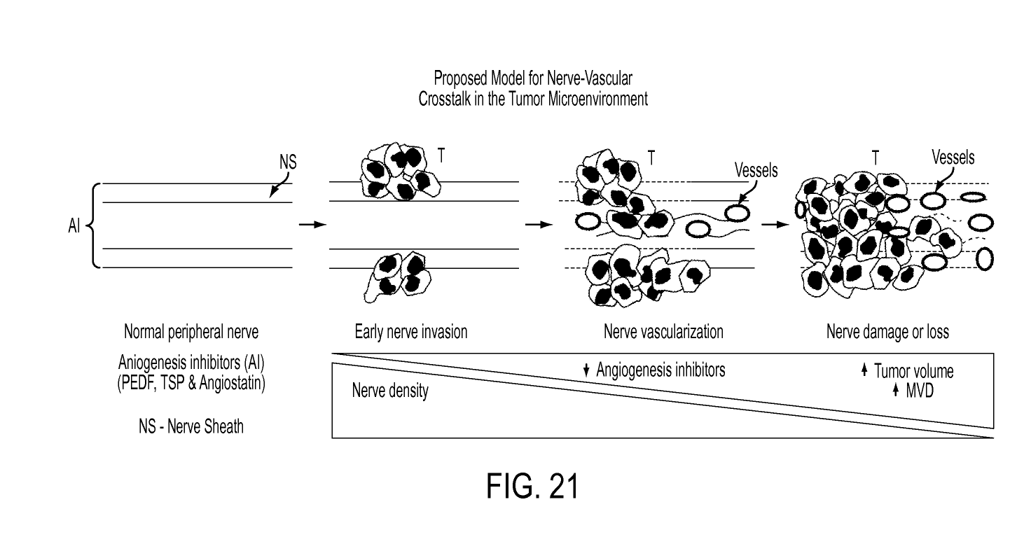

FIG. 21 shows a proposed model for neural-vascular crosstalk in the tumor microenvironment. The tumor growth process proceeds from early tumor invasion to nerve vascularization and eventually to nerve damage or loss. As nerve density decreases, the concentration of angiogenesis inhibitors normally produced by nervous tissue also decreases. The angiogenesis inhibitors include PEDF, TSP-1, and angiostatin. As nerve density decreases and tumor growth progresses, there is an increase in tumor volume and tumor microvessel density (MVD). In FIG. 21, “NS” designates the nerve sheath, “AI” designates the angiogenesis inhibitors, and “T” designates the tumor.

Potential delivery systems and schemes for delivery are shown in FIGS. 5 and 7, above.

To summarize the findings reported herein: (1) Neural stem cells are currently used in the setting of neurodegenerative diseases—to date, they have not been recognized as having utility against non-neural tumors. (2) Anti-angiogenic activity of neural stem cells has not been reported. (3) Since neural stem cells appear to act as an intrinsic barrier to tumor growth, this suggests that denervation during surgery appears to be tumor-promoting with respect to the tumor microenvironment.

The foregoing example clearly establishes that this method of treating cancer is particularly applicable to prostate cancer.

Accordingly, the present invention provides a novel method of cancer therapy employing neural stem cells applied with a stem-cell-rich bioadhesive.

In general, such a method comprises the steps of:

(1) harvesting neural stem cells;

(2) culturing neural stem cells under conditions such that the cells proliferate while retaining their ability to differentiate; and

(3) applying a biocompatible adhesive to a post-surgical site of a malignancy; and

(4) following the application of the biocompatible adhesive to the post-surgical site of the malignancy, applying the cultured neural stem cells to the post-surgical site of the malignancy to remove residual tumor cells and to induce endothelial cell apoptosis.

Typically, the step of harvesting neural stem cells is performed by harvesting the stem cells from bone marrow. An alternative source of the neural stem cells is adipose tissue. These stem cells are adult stem cells rather than embryonic stem cells and obtaining them does not involve any manipulations to or destruction of embryos. Typically, these stem cells from bone marrow or adipose tissue are taken from the patient to be treated, thereby avoiding any problems with tissue matching and potential rejection and immune responses that might impair efficacy of the treatment.

In another alternative, neural stem cells suitable for use in methods according to the present invention can be derived from the patient's own hematopoietic stem cells or from cells from peripheral nerve biopsies (Schwann cells). These sources can be used to derive neural stem cells by the following techniques: For the derivation of neural stem cells from Schwann cells, Schwann cells were cultured from rat sciatic nerves as described in J. P. Brockes et al., “Studies on Cultured Rat Schwann Cells. I. Establishment of Purified Populations from Cultures of Peripheral Nerve,” Brain Res. 165: 105-118 (1979). Briefly, nerves were removed from 3-day-old rat pups, digested for 2 hours in 0.03% collagenase (Serva) at 37° C., and triturated thoroughly to achieve dissociation. Cells were maintained as monolayers in low glucose DMEM supplemented with 10% FBS, 100 units/ml penicillin and 100 mg/ml streptomycin at 37° C. in a humid atmosphere of 10% CO₂/90% air. Contaminating fibroblasts were inhibited by 72 hours treatment with 10 mM cytosine arabinoside. Purity of Schwann cell cultures were confirmed by staining with antibody raised to S-100 protein (DAKO), and only cultures that contained >95% S-100 positive cells were used in experiments. An improved method is described in G. T. Casella et al., “Improved Method for Harvesting Human Schwann Cells from Mature Peripheral Nerve and Expansion in Vivo,” Glia 17: 327-338 Alternatively, umbilical cord stroma-derived stem cells are harvested by enzymatic dissociation of fragments of umbilical cord into single cells and cultured by standard cell culture techniques. The culture medium is enriched with bFGF and PEDF.

The step of harvesting the neural stem cells is typically performed by using a high efficiency cell sorter to retrieve the neural stem cells. Such high efficiency cell sorters are known in the art. Such cell sorters typically sort cells by fluorescence-activated cell sorting (FACS), typically employing fluorochrome-conjugated antibodies. The FACS sorting employs one or more markers selected from the group consisting of nestin, PAX6, CD44, SOX2, RC2, BLBP, and GLAST. Nestin is a type VI intermediate filament protein. PAX6 (paired box gene 6) is a gene whose product exerts control over neural development. CD44 is a cell-surface glycoprotein involved in cell-cell interaction, cell adhesion, and migration. SOX2 ((sex determining region Y)-box 2) is a transcription factor active in stem cells. RC2 is an antigen similar to, if not identical to, nestin. BLBP is brain lipid binding protein. GLAST is glutamate aspartate transporter. Methods for performing such stem cell sorting employing commercially-available FACS apparatus are known in the art, and are described, for example, in J. Pruszak et al., “Markers and Methods for Cell Sorting of Human Embryonic Stem Cell-Derived Neural Cell Populations,” Stem Cells 25: 2257-2268 (2007), incorporated herein by this reference. Other separation methods, such as magnetic separation, are also known in the art. Methods for harvesting and propagating neural stem cells are known in the art, and are described, for example, in United States Patent Application Publication No. 2010/0323444 by Steindler et al., United States Patent Application Publication No. 2005/0009742 by Bertilsson et al., United States Patent Application Publication No. 2005/0009847 by Bertilsson et al., United States Patent Application Publication No. 2005/0032702 by Eriksson, United States Patent Application Publication No. 2005/0031538 by Steindler et al., United States Patent Application Publication No. 2005/0004046 by van Praag et al., United States Patent Application Publication No. 2004/0254152 by Monje et al., United States Patent Application Publication No. 2004/0229291 by Zhou et al., and United States Patent Application Publication No. 2004/0185429 by Kelleher-Andersson et al., all of which are incorporated herein in their entirety by this reference.

The isolated neural stem cells are then cultured. Their proliferation is stimulated by treatment with bFGF (basic fibroblast growth factor). Possible other growth factors include EGF, TGF-α, and aFGF; see United States Patent Application Publication No. 2010/0239541 by Johe et al., incorporated herein in its entirety by this reference. The culture medium used is preferably DMEM/F12 (Invitrogen)) supplemented with N2 (Invitrogen), B27 (Invitrogen), 2 μg/ml heparin, 100 U/ml penicillin, and 100 μg/ml streptomycin. Conventional culture media used for neural stem cells are known in the art and are described, for example, in United States Patent Application Publication No. 2011/0020931 by Onodera et al., and in United States Patent Application Publication No. 2010/0323444 by Steindler et al. and include DMEM/F12 medium (Invitrogen). Other growth-promoting additives, such as insulin, transferrin, selenium (i.e., sodium selenite), putrescine, or progesterone can be included. Additional growth promoting factors such as pituitary extract, antibiotics, and fetal calf serum can also be included.

The next step is the application of a biocompatible adhesive to the post-surgical site of the malignancy. A number of biocompatible adhesives are known in the art.

One particularly preferred biocompatible adhesive is a DOPA-substituted polyethylene glycol (PEG) polymer that has been designed to mimic the action of the adhesive used to hold mussels to their site of attachment. A general structural formula for such a mussel-mimetic biocompatible adhesive is shown below.

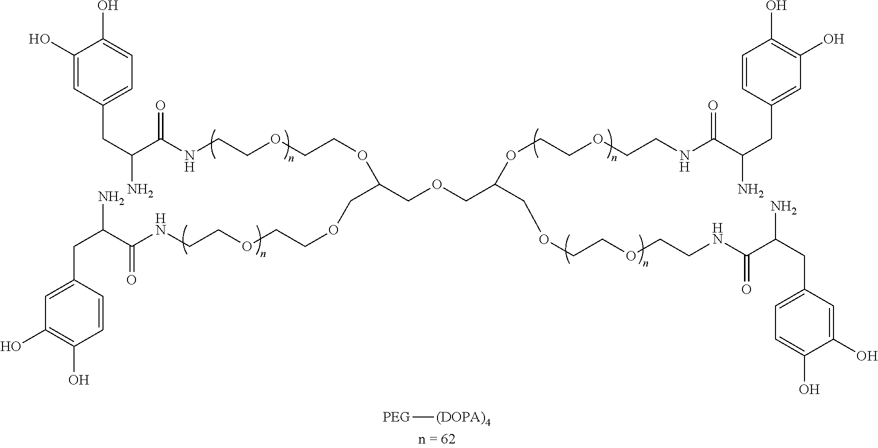

In general, such a structure comprises a core consisting of a branched PEG molecule whose four arms are each chemically derivatized with DOPA moieties. These mussel-mimetic adhesives are described in G. Bilic et al., “Injectable Candidate Sealants for Fetal Membrane Repair: Bonding and Toxicity in Vitro,” Am. J. Obstet. Gynecol. 202: 85.e1-9 (2010); C. E. Brubaker et al., “Biological Performance of Mussel-Inspired Adhesive in Extrahepatic Islet Transplantation,” Biomaterials 31: 420-427 (2010); H. Lee et al., “Facile Conjugation of Molecules onto Surfaces via Mussel Adhesive Protein Inspired Coatings,” Adv. Mater. 21: 431-434 (2009); N. Holten-Andersen et al., “pH-Induced Metal-Ligand Cross-Links Inspired by Mussel Yield Self-Healing Polymer Networks with Near-Covalent Elastic Moduli,” Proc. Natl. Acad. Sci USA 108: 2651-2655 (2011); H. Lee et al., “Mussel-Inspired Surface Chemistry for Multifunctional Coatings,” Science 318: 426-430 (2007); S. H. Ku et al., “Spatial Control of Cell Adhesion and Patterning Through Mussel-Inspired Surface Modification by Polydopamine,” Langmuir Lett. 26: 15104-15108 (2010); and S. Kim & C. B. Park, “Dopamine-Induced Mineralization of Calcium Carbonate Vaterite Microspheres,” Langmuir Lett. 26: 14730-14736 (2010) In such adhesives, n typically is from about 20 to about 100, preferably from about 40 to about 80, more preferably 62. Derivatives of these polydopamine adhesives can alternatively be used, such as derivatives in which one or more of the hydrogen atoms in the —OH groups of the polydopamine are replaced by lower alkyl groups, such as C₁-C₆ alkyl, to produce alkoxy groups.

The interaction between neural stem cells and polydopamine coated surfaces is shown in FIGS. 9 and 10. FIG. 9 shows that neurospheres are visible on polydopamine. The left panel of FIG. 9 is a control; the right panel of FIG. 9 shows that neurospheres form on polydopamine. FIG. 10 shows an attached neurosphere (NS) cultured with pancreatic adenocarcinoma (Panc-1) cells showing the effect of the neurospheres in promoting regression of the Panc-1 cells. The formula for polydopamine is [(HO)₂C₆H₃CH₂CH₂NH₂]ₙ.

The structure of dopamine is shown below in Formula (I)

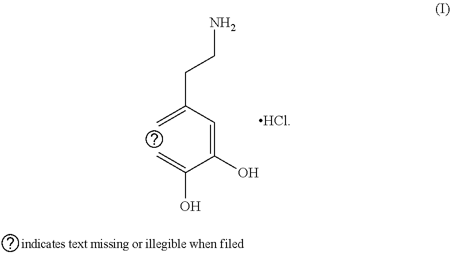

Dopamine autopolymerizes spontaneously in mildly alkaline solution (pH 8.5) to polydopamine. This is described in S. H. Ku et al., “Spatial Control of Cell Adhesion and Patterning Through Mussel-Inspired Surface Modification by Polydopamine,” Langmuir Lett. 26: 15104-15108 (2010), incorporated herein by this reference. This is shown in Reaction Scheme (I), below.

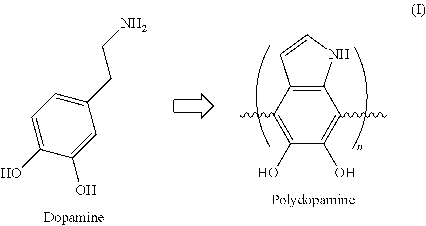

Similarly, Reaction Scheme (II), below, shows the further derivatization of polydopamine.

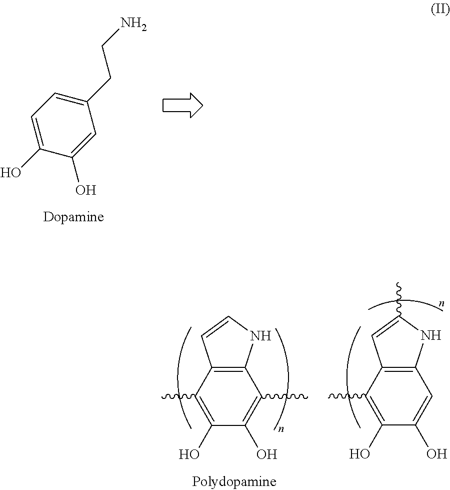

Dopamine hydrochloride (3-hydroxytyramine hydrochloride) at 2 mg/ml is dissolved in 10 mM Tris buffer (pH 8.5) and filter sterilized. Petri dishes, tissue culture dishes or coverslips used for cell culture are coated by flooding the surface with the polydopamine solution and left overnight protected from light in the incubator at 37° C. The polydopamine coating is rinsed with sterile double distilled water (×3) and the surface allowed to dry. The coated dishes can be used right away or kept at 37° C. in the incubator to be used in the next four weeks.

Alternatively, other biocompatible adhesives can be employed, such as fibrin glues, alkyl-α-cyanoacrylate glues, and other biocompatible adhesives known in the art. Additional biocompatible adhesives are disclosed in U.S. Pat. No. 7,883,693 to Sehl et al., U.S. Pat. No. 7,858,079 to Hadba et al., and U.S. Pat. No. 7,501,133 to McNally-Heintzelman et al.

The adhesive is applied to the post-surgical site of the malignancy by conventional techniques for application of adhesives or other treatment materials to post-surgical sites. For example, and not by way of limitation, a conventional double-barreled syringe can be used.

Following the application of the adhesive to the post-surgical site of the malignancy, the neural stem cells are also applied to the post-surgical site of the malignancy so that the neural stem cells are in contact with and are entrapped by the adhesive. The neural stem cells induce endothelial cell apoptosis and release anti-angiogenic proteins into the circulation to prevent tumor cell angiogenesis from occurring. These anti-angiogenic proteins include PEDF (pigment epithelium-derived factor), TSP (thrombospondin), and angiostatin.

As indicated above, the interaction between the neural stem cells and any residual tumor cells exerts an anti-angiogenic effect on the tumor cells. This prevents the converse effect, namely the effect of tumor cells in causing denervation. This converse effect is shown in FIG. 8. In FIG. 8, a model of tumor-nerve-vessel interactions during perineural invasion and cancer progression is presented. Tumor cells (T) are initially confined to the region of the neural sheath (NS). With a permissive microenvironment, this barrier is breached, and the tumor invades the nerve itself which is accompanied by intraneural vascularization and subsequent nerve damage or loss. The interaction between the neural stem cells and any residual tumor cells exerts an anti-angiogenic effect and prevents denervation in addition to blocking replication of any residual tumor cells.

Typically, the neural stem cells are applied with survival factors. These survival factors include pigment epithelium derived factor and propranolol.

Methods according to the present invention can further comprise the additional step of administering a therapeutically effective quantity of an additional anti-neoplastic therapeutic agent to the patient. Examples of suitable anti-neoplastic therapeutic agents are listed below; however, the present invention encompasses the use of other anti-neoplastic agents known in the art. For anti-neoplastic agents known in the art, one of ordinary skill in the art can determine appropriate dosages, frequencies of administration, and durations of administration.

Mechlorethamine is a nitrogen mustard that has anti-neoplastic activity against Hodgkin's disease and non-Hodgkin's lymphomas. Cyclophosphamide and ifosfamide are nitrogen mustards that have anti-neoplastic activity against acute and chronic lymphocytic leukemia, Hodgkin's disease, non-Hodgkin's lymphomas, multiple myeloma, neuroblastoma, breast cancer, ovarian cancer, lung cancer, Wilms' tumor, cervical cancer, testicular cancer, and soft tissue sarcomas. Melphalan is a nitrogen mustard that has anti-neoplastic activity against multiple myeloma, breast cancer, and ovarian cancer. Chlorambucil is a nitrogen mustard that has anti-neoplastic activity against chronic lymphocytic leukemia, primary macroglobulinemia, Hodgkin's disease, and non-Hodgkin's lymphomas. Hexamethylmelamine is an alkylating agent that has anti-neoplastic activity against ovarian cancer. Thiotepa is an alkylating agent that has anti-neoplastic activity against bladder cancer, breast cancer, and ovarian cancer. Busulfan is an alkyl sulfonate that has anti-neoplastic activity against chronic granulocytic leukemia. Carmustine is a nitrosourea that has anti-neoplastic activity against, Hodgkin's disease, non-Hodgkin's lymphomas, primary brain tumors, multiple myeloma, and malignant melanoma. Streptozocin is a nitrosourea that has anti-neoplastic activity against malignant pancreatic insulinoma and malignant carcinoid. Dacarbazine is a triazene that has anti-neoplastic activity against malignant melanoma, Hodgkin's disease, and soft-tissue sarcomas. Temozolomide is a triazene that has anti-neoplastic activity against glioma and malignant melanoma. Methotrexate is a folic acid analogue that has anti-neoplastic activity against acute lymphocytic leukemia, choriocarcinoma, mycosis fungoides, breast cancer, head and neck cancer, lung cancer, and osteogenic sarcoma. 5-Fluorouracil is a pyrimidine analogue antimetabolite that has anti-neoplastic activity against breast cancer, colon cancer, stomach cancer, pancreatic cancer, ovarian cancer, head and neck cancer, and urinary bladder cancer. Cytarabine is a pyrimidine analogue antimetabolite that has anti-neoplastic activity against acute granulocytic leukemia and acute lymphocytic leukemia. Gemcitabine is a pyrimidine analogue antimetabolite that has anti-neoplastic activity against pancreatic cancer and ovarian cancer. 6-Mercaptopurine is a purine analogue antimetabolite that has anti-neoplastic activity against acute lymphocytic leukemia, acute granulocytic leukemia, and chronic granulocytic leukemia. 6-Thioguanine is a purine analogue antimetabolite that has anti-neoplastic activity against acute lymphocytic leukemia, acute granulocytic leukemia, and chronic granulocytic leukemia. Pentostatin is a purine analogue antimetabolite that has anti-neoplastic activity against hairy cell leukemia, mycosis fungoides, chronic lymphocytic leukemia, and small cell leukemia. Vinblastine is a vinca alkaloid that has anti-neoplastic activity against Hodgkin's disease, non-Hodgkin's lymphoma, breast cancer, and testicular cancer. Vincristine is a vinca alkaloid that has anti-neoplastic activity against acute lymphocytic leukemia, neuroblastoma, Wilms' tumor, rhabdomyosarcoma, Hodgkin's disease, non-Hodgkin's lymphoma, and small-cell lung cancer. Paclitaxel is a taxane that has anti-neoplastic activity against ovarian cancer, lung cancer, breast cancer, and head and neck cancer. Docetaxel is a taxane that has anti-neoplastic activity against ovarian cancer, lung cancer, breast cancer, and head and neck cancer. Topotecan is a camptothecin that has anti-neoplastic activity against ovarian cancer and small-cell lung cancer. Irinotecan is a camptothecin that has anti-neoplastic activity against colon cancer. Dactinomycin is an antibiotic that has anti-neoplastic activity against choriocarcinoma, Wilms' tumor, rhabdomyosarcoma, testicular cancer, and Kaposi's sarcoma. Daunorubicin is an antibiotic that has anti-neoplastic activity against acute granulocytic and acute lymphocytic leukemias. Doxorubicin is an antibiotic that has anti-neoplastic activity against soft-tissue sarcomas, osteogenic sarcomas, other sarcomas, Hodgkin's disease, non-Hodgkin's lymphoma, acute leukemias, breast cancer, genitourinary tract cancer, thyroid cancer, lung cancer, gastric cancer, and neuroblastoma. Bleomycin is an antibiotic that has anti-neoplastic activity against testicular cancer, head and neck cancer, skin cancer, esophageal cancer, lung cancer, genitourinary tract cancer, Hodgkin's disease, and non-Hodgkin's lymphoma. Mitomycin C is an antibiotic that has anti-neoplastic activity against gastric cancer, cervical cancer, colon cancer, breast cancer, pancreatic cancer, bladder cancer, and head and neck cancer. L-asparaginase is an enzyme that has anti-neoplastic activity against acute lymphocytic leukemia. Interferon-alfa is a biological response modifier that has anti-neoplastic activity against hairy cell leukemia, Kaposi's sarcoma, malignant melanoma, carcinoid, renal cell cancer, ovarian cancer, bladder cancer, non-Hodgkin's lymphoma, mycosis fungoides, multiple myeloma, and chronic granulocytic leukemia. Interleukin-2 is a biological response modifier that has anti-neoplastic activity against malignant melanoma and renal cell cancer. Cisplatin is a platinum coordination complex that has anti-neoplastic activity against testicular cancer, ovarian cancer, bladder cancer, head and neck cancer, lung cancer, thyroid cancer, cervical cancer, endometrial cancer, neuroblastoma, and osteogenic sarcoma. Carboplatin is a platinum coordination complex that has anti-neoplastic activity against testicular cancer, ovarian cancer, bladder cancer, head and neck cancer, lung cancer, thyroid cancer, cervical cancer, endometrial cancer, neuroblastoma, and osteogenic sarcoma. Mitoxantrone is an anthracenedione that has anti-neoplastic activity against acute granulocytic leukemia, breast cancer, and prostate cancer. Hydroxyurea is a substituted urea that has anti-neoplastic activity against chronic granulocytic leukemia, polycythemia vera, essential thrombocytosis, and malignant melanoma. N-methylhydrazine is a substituted hydrazine that has anti-neoplastic activity against Hodgkin's disease. Mitotane is an adrenocortical suppressant that has anti-neoplastic activity against adrenal cortex cancer. Aminoglutethimide is an adrenocortical suppressant that has anti-neoplastic activity against breast cancer. Imatinib is a tyrosine kinase inhibitor that has anti-neoplastic activity against chronic myelocytic leukemia. Prednisone is an adrenocorticosteroid that has anti-neoplastic activity against acute lymphocytic leukemia, chronic lymphocytic leukemia, Hodgkin's disease, non-Hodgkin's lymphoma, and breast cancer. Prednisolone is an adrenocorticosteroid that has anti-neoplastic activity against acute lymphocytic leukemia, chronic lymphocytic leukemia, Hodgkin's disease, non-Hodgkin's lymphoma, and breast cancer. Methylprednisolone is an adrenocorticosteroid that has anti-neoplastic activity against acute lymphocytic leukemia, chronic lymphocytic leukemia, Hodgkin's disease, non-Hodgkin's lymphoma, and breast cancer. Dexamethasone is an adrenocorticosteroid that has anti-neoplastic activity against acute lymphocytic leukemia, chronic lymphocytic leukemia, Hodgkin's disease, non-Hodgkin's lymphoma, and breast cancer. Betamethasone is an adrenocorticosteroid that has anti-neoplastic activity against acute lymphocytic leukemia, chronic lymphocytic leukemia, Hodgkin's disease, non-Hodgkin's lymphoma, and breast cancer. Triamcinolone is an adrenocorticosteroid that has anti-neoplastic activity against acute lymphocytic leukemia, chronic lymphocytic leukemia, Hodgkin's disease, non-Hodgkin's lymphoma, and breast cancer. Hydroxyprogesterone caproate is a progestin that has anti-neoplastic activity against endometrial cancer and breast cancer. Medroxyprogesterone acetate is a progestin that has anti-neoplastic activity against endometrial cancer and breast cancer. Megestrol acetate is a progestin that has anti-neoplastic activity against endometrial cancer and breast cancer. Diethylstilbestrol is an estrogen that has anti-neoplastic activity against breast cancer and prostate cancer. Ethinyl estradiol is an estrogen that has anti-neoplastic activity against breast cancer and prostate cancer. Tamoxifen is an antiestrogen that has anti-neoplastic activity against breast cancer. Anastrozole is an antiestrogen that has anti-neoplastic activity against breast cancer. Testosterone propionate is an androgen that has anti-neoplastic activity against breast cancer. Fluoxymesterone is an androgen that has anti-neoplastic activity against breast cancer. Flutamine is an antiandrogen that has anti-neoplastic activity against prostate cancer. Leuprolide is a gonadotropin-releasing hormone analogue that has anti-neoplastic activity against prostate cancer. Trastuzumab is a monoclonal antibody that has anti-neoplastic activity against breast cancer. Rituximab is a monoclonal antibody that has anti-neoplastic activity against non-Hodgkin's lymphoma. Alemtuzumab is a monoclonal antibody that has anti-neoplastic activity against chronic lymphocytic leukemia. Bevacizumab is a monoclonal antibody that has anti-neoplastic activity against colorectal cancer. Cetuximab is a monoclonal antibody that has anti-neoplastic activity against colorectal cancer and head and neck cancer. Gemtuzumab is a monoclonal antibody that has anti-neoplastic activity against acute myelocytic leukemia. Ibritumomab is a monoclonal antibody that has anti-neoplastic activity against non-Hodgkin's lymphoma. Panitumumab is a monoclonal antibody that has anti-neoplastic activity against colorectal cancer. Tositumomab is a monoclonal antibody that has anti-neoplastic activity against non-Hodgkin's lymphoma.

Another group of anti-neoplastic agents suitable for use in methods according to the present invention is interferons. Interferon works in a different way toward cancer cells than it does toward viruses and there are numerous pathways that interferon activates to help treat cancers. It has an antiproliferative effect on tumor cells, it stimulates the tumor cells to change their surfaces so that they are recognized by the immune system as abnormal cells, and it blocks the growth of new blood vessels and helps cut off the supply of nutrients. At this time there are 12 identified interferon alphas.

Other anti-neoplastic agents are known in the art and can be employed in methods according to the present invention. For example, additional anti-neoplastic agents are disclosed in U.S. Pat. No. 7,329,638 to Yang et al., incorporated herein by this reference. These additional anti-neoplastic agents include: (1) epidermal growth factor receptor (EGFR) kinase inhibitors; (2) vascular growth factor receptor (VGFR) kinase inhibitors; (3) fibroblast growth factor receptor (FGFR) kinase inhibitors; (4) platelet derived growth factor receptor (PDGF) kinase inhibitors; (5) Bcr-Ab1 kinase inhibitors such as STI-571; (6) antisense molecules; (6) anti-estrogens such as raloxifene; (7) anti-androgens such as flutamide, bicalutamide, finasteride, aminoglutethimide, and ketoconazole; (8) non-steroidal anti-inflammatory drugs including cyclooxygenase-2 (COX-2) inhibitors; (9) additional chemotherapeutic drugs including fludarabine, Mylotarg, and etoposide (a topoisomerase inhibitor); (10) cellular signaling molecules; (11) ceramides; (12) cytokines; (13) staurosprine; (14) altretamine; (15) capecitabine; (16) cladribine; (17) idarubicin; (18) vinorelbine; (19) geranylgeraniol {3,7,11,15-tetramethyl-2,6,10,14-hexadecatraen-1-ol); (20) pro-apoptotic Bcl-2 family proteins including Bax, Bak, Bid, and Bad); (21) photodynamic compounds such as Photofrin (II), ruthenium red compounds (e.g., Ru-diphenyl-phenanthroline and Tris(1-10-phenanthroline)ruthenium(II)chloride), tin ethyl etiopurpurin, protoporphyrin IX, chloroaluminum phthalocyanine, tetra(M-hydroxyphenyl)chlorin)); (22) radiodynamic (i.e., scintillating) compounds such as NaI-125, 2,5-diphenyloxazole (PPO); 2-(4-biphenyl)-6-phenylbenzoxazole; 2,5-bis-(5′-tert-butylbenzoxazoyl-[2′])thiophene; 2-(4-t-butylphenyl)-5-(4-biphenylyl)-1,3,4-oxadiazole; 1,6-diphenyl-1,3,5-hexatriene; trans-p,p′-diphenylstilbene; 2-(1-naphthyl)-5-phenyloxazole; 2-phenyl-5-(4-biphenylyl)-1,3,4-oxadiazole; p-terphenyl; and 1,1,4,4-tetraphenyl-1,3-butadiene); (23) lomustine; (24) semustine; (25) hydroxymethylmelamine; (26) plicamycin; (27) azathioprine; (28) 2-chlorodeoxyadenosine; (29) 5-fluorodeoxyuridine; and (30) batimistat, as well as other compounds known in the art.

Yet another category of anti-neoplastic agents that could be used as additional therapeutic agents in methods according to the present invention are anti-angiogenic agents, such as those described in U.S. Pat. No. 7,867,975 to O'Reilly et al., U.S. Pat. No. 7,524,811 to Folkman et al., U.S. Pat. No. 7,495,089 to O'Reilly et al., U.S. Pat. No. 7,365,159 to O'Reilly et al., U.S. Pat. No. 7,332,523 to Satchi-Fainaro et al., U.S. Pat. No. 7,179,608 to O'Reilly et al., U.S. Pat. No. 7,157,556 to Pirie-Shepherd et al., U.S. Pat. No. 6,949,584 to Satchi-Fainaro et al., U.S. Pat. No. 6,949,511 to O'Reilly et al., U.S. Pat. No. 6,794,995 to O'Reilly et al., U.S. Pat. No. 6,630,438 to O'Reilly et al., U.S. Pat. No. 6,607,724 to O'Reilly et al., U.S. Pat. No. 6,346,510 to O'Reilly et al., U.S. Pat. No. 6,086,065 to Folkman et al., U.S. Pat. No. 6,024,688 to Folkman et al., and U.S. Pat. No. 6,017,954 to Folkman et al., all of which are incorporated herein by this reference.

In addition, the salts, solvates, analogues, congeners, bioisosteres, hydrolysis products, metabolites, precursors, and prodrugs of the therapeutic agents described above could be used in methods according to the present invention.

In the case of salts, it is well known that organic compounds, including compounds having activities suitable for methods according to the present invention, have multiple groups that can accept or donate protons, depending upon the pH of the solution in which they are present. These groups include carboxyl groups, hydroxyl groups, amino groups, sulfonic acid groups, and other groups known to be involved in acid-base reactions. The recitation of a compound or analogue includes such salt forms as occur at physiological pH or at the pH of a pharmaceutical composition unless specifically excluded. Pharmacologically acceptable salts include, but are not limited to, the salts described below.

Similarly, prodrug esters can be formed by reaction of either a carboxyl or a hydroxyl group on compounds or analogues suitable for methods according to the present invention with either an acid or an alcohol to form an ester. Typically, the acid or alcohol includes a lower alkyl group such as methyl, ethyl, propyl, isopropyl, butyl, isobutyl, and tertiary butyl. These groups can be substituted with substituents such as hydroxy, or other substituents. Such prodrugs are well known in the art and need not be described further here. The prodrug is converted into the active compound by hydrolysis of the ester linkage, typically by intracellular enzymes. Other suitable groups that can be used to form prodrug esters are well known in the art. For example prodrugs can include amides prepared by reaction of the parent acid compound with a suitable amine. In some cases it is desirable to prepare double ester type prodrugs such as (acyloxy) alkyl esters or ((alkoxycarbonyl)oxy)alkyl esters. Suitable esters as prodrugs include, but are not necessarily limited to, methyl, ethyl, propyl, isopropyl, n-butyl, isobutyl, tert-butyl, morpholinoethyl, and N,N-diethylglycolamido. Methyl ester prodrugs may be prepared by reaction of the acid form of a compound having a suitable carboxylic acid group in a medium such as methanol with an acid or base esterification catalyst (e.g., NaOH, H₂ SO₄). Ethyl ester prodrugs are prepared in similar fashion using ethanol in place of methanol. Morpholinylethyl ester prodrugs may be prepared by reaction of the sodium salt of a suitable compound (in a medium such as dimethylformamide) with 4-(2-chloroethyl)morphine hydrochloride (available from Aldrich Chemical Co., Milwaukee, Wis. USA.

Pharmaceutically acceptable salts include inorganic or organic acid salts such as hydrochloride, hydrobromide, hydroiodide, sulfate, phosphate, fumarate, maleate, acetate, citrate, lactate, tartrate, sulfamate, malonate, succinate, methanesulfonate, ethanesulfonate, benzenesulfonate, p-toluenesulfonate, cyclohexylsulfamate, quinate, formate, cinnamate, picrate, propionate, succinate, glycolate, gluconate, ascorbate, benzoate, anthranilate, mesylate, p-hydroxybenzoate, phenylacetate, mandelate, embonate, pantothenate, 2-hydroxyethanesulfonate, sulfanilate, stearate, cyclohexylaminosulfonate, algenate, β-hydroxybutyrate, salicylate, galactarate, galacturonate, and other suitable salts. Such salts can be derived using acids such as hydrochloric acid, sulfuric acid, phosphoric acid, sulfamic acid, acetic acid, citric acid, lactic acid, tartaric acid, malonic acid, methanesulfonic acid, ethanesulfonic acid, benzenesulfonic acid, p-toluenesulfonic acid, cyclohexylsulfamic acid, and quinic acid, as well as the other corresponding acids listed above.

Pharmaceutically acceptable salts also include salts with bases such as alkali metal salts such as sodium or potassium, as well as pyridine salts, ammonium salts, piperazine salts, diethylamine salts, nicotinamide salts, calcium salts, magnesium salts, zinc salts, lithium salts, methylamino salts, triethylamino salts, dimethylamino salts, N,N′-dibenzylethylenediamino salts, choline salts, diethanolamine salts, chloroprocaine salts, ethylenediamino salts, meglumine salts, procaine salts, and tris(hydroxymethyl) aminomethane salts. Such salts can be derived using the appropriate base.

Additionally, therapeutic agents as described above could be coupled to a targeting moiety such as a hormone, a ligand for a cellular receptor, or a monoclonal antibody. Such coupling is typically performed by reactions such as those described below. The result is a covalent conjugate including the therapeutic agent and the targeting moiety.

Suitable reagents for cross-linking many combinations of functional groups are known in the art. For example, electrophilic groups can react with many functional groups, including those present in proteins or polypeptides. Various combinations of reactive amino acids and electrophiles are known in the art and can be used. For example, N-terminal cysteines, containing thiol groups, can be reacted with halogens or maleimides. Thiol groups are known to have reactivity with a large number of coupling agents, such as alkyl halides, haloacetyl derivatives, maleimides, aziridines, acryloyl derivatives, arylating agents such as aryl halides, and others. These are described in G. T. Hermanson, “Bioconjugate Techniques” (Academic Press, San Diego, 1996), pp. 146-150, incorporated herein by this reference. The reactivity of the cysteine residues can be optimized by appropriate selection of the neighboring amino acid residues. For example, a histidine residue adjacent to the cysteine residue will increase the reactivity of the cysteine residue. Other combinations of reactive amino acids and electrophilic reagents are known in the art. For example, maleimides can react with amino groups, such as the ε-amino group of the side chain of lysine, particularly at higher pH ranges. Aryl halides can also react with such amino groups. Haloacetyl derivatives can react with the imidazolyl side chain nitrogens of histidine, the thioether group of the side chain of methionine, and the ε-amino group of the side chain of lysine. Many other electrophilic reagents are known that will react with the ε-amino group of the side chain of lysine, including, but not limited to, isothiocyanates, isocyanates, acyl azides, N-hydroxysuccinimide esters, sulfonyl chlorides, epoxides, oxiranes, carbonates, imidoesters, carbodiimides, and anhydrides. These are described in G. T. Hermanson, “Bioconjugate Techniques” (Academic Press, San Diego, 1996), pp. 137-146, incorporated herein by this reference. Additionally, electrophilic reagents are known that will react with carboxylate side chains such as those of aspartate and glutamate, such as diazoalkanes and diazoacetyl compounds, carbonydilmidazole, and carbodiimides. These are described in G. T. Hermanson, “Bioconjugate Techniques” (Academic Press, San Diego, 1996), pp. 152-154, incorporated herein by this reference. Furthermore, electrophilic reagents are known that will react with hydroxyl groups such as those in the side chains of serine and threonine, including reactive haloalkane derivatives. These are described in G. T. Hermanson, “Bioconjugate Techniques,” (Academic Press, San Diego, 1996), pp. 154-158, incorporated herein by this reference. In another alternative embodiment, the relative positions of electrophile and nucleophile (i.e., a molecule reactive with an electrophile) are reversed so that, for example, if a protein is to be coupled to another molecule, the protein to be coupled has an amino acid residue with an electrophilic group that is reactive with a nucleophile and the molecule with which the protein to be coupled includes therein a nucleophilic group. This includes the reaction of aldehydes (the electrophile) with hydroxylamine (the nucleophile), described above, but is more general than that reaction; other groups can be used as electrophile and nucleophile. Suitable groups are well known in organic chemistry and need not be described further in detail.

Additional combinations of reactive groups for cross-linking are known in the art. For example, amino groups can be reacted with isothiocyanates, isocyanates, acyl azides, N-hydroxysuccinimide (NHS) esters, sulfonyl chlorides, aldehydes, glyoxals, epoxides, oxiranes, carbonates, alkylating agents, imidoesters, carbodiimides, and anhydrides. Thiol groups can be reacted with haloacetyl or alkyl halide derivatives, maleimides, aziridines, acryloyl derivatives, acylating agents, or other thiol groups by way of oxidation and the formation of mixed disulfides. Carboxy groups can be reacted with diazoalkanes, diazoacetyl compounds, carbonyldiimidazole, carbodiimides. Hydroxyl groups can be reacted with epoxides, oxiranes, carbonyldiimidazole, N,N′-disuccinimidyl carbonate, N-hydroxysuccinimidyl chloroformate, periodate (for oxidation), alkyl halogens, or isocyanates. Aldehyde and ketone groups can react with hydrazines, reagents forming Schiff bases, and other groups in reductive amination reactions or Mannich condensation reactions. Still other reactions suitable for cross-linking reactions are known in the art. In some cases, it may be desirable to introduce a specific functional group that can subsequently be cross-linked. Such functional groups that can be introduced for cross-linking purposes can include, for example, sulfhydryl groups, carboxylate groups, primary amine groups, aldehyde groups, and hydrazide groups. Such cross-linking reagents and reactions, including the introduction of suitable functional groups for cross-linking, are described in G. T. Hermanson, “Bioconjugate Techniques” (Academic Press, San Diego, 1996), incorporated herein in its entirety by this reference.

The targeting moiety and the therapeutic agent can be conjugated directly, or through a linker. Suitable linkers are known in the art and include peptides and polyethylene glycol chains. Suitable linkers are described, for example, in United States Patent Application Publication No. 2008/0213249 by Sinha et al. and in United States Patent Application Publication No. 2007/0187499 by Barbas, both of which are incorporated herein by this reference.

Still other anti-neoplastic agents can be used as additional therapeutic agents in methods according to the present invention.

In yet another alternative, methods according to the present invention can further comprise the additional step of administering an effective quantity of anti-neoplastic ionizing radiation. The use of ionizing radiation to treat malignancies is well known and is described, for example, in R. G. Parker & H. R. Withers, “Principles of Radiation Oncology” in C. M. Haskell, Cancer Treatment (5^{th} ed., W.B. Saunders Co., Philadelphia, 2001), ch. 7, pp. 52-61, incorporated herein by this reference. Methods of ionizing radiation treatment include treatment with X-rays or proton beams, brachytherapy with radioisotopes such as cesium-137, cobalt-60, indium-192, iodine-125, palladium-103, and ruthenium-106, and radioisotope therapy with radioisotopes such as iodine-131, lutetium-177, yttrium-90, strontium-89, and samarium-153, which can be conjugated to targeting moieties such as hormones, receptor targets, or monoclonal antibodies. Such conjugation methods are well known in the art and need not be described further in detail. Other methods for radiation therapy are known in the art.

Methods according to the present invention can be used for the treatment of malignancies in humans as well as in socially or economically important non-human mammalian species such as, but not limited to, dogs, cats, horses, pigs, goats, cattle, sheep, mules, donkeys, buffalo, or other socially or economically important non-human mammalian species. Unless specifically stated, methods according to the present invention are not limited to the treatment of malignancies in humans.

As used herein, terms such as “treatment,” “treating,” and similar terminology includes the administration of compounds or agents to a subject to prevent or delay the onset of the symptoms, complications, or biochemical indicia of a disease, condition, or disorder (e.g., a malignancy), alleviating the symptoms of the disease, condition, or disorder, or arresting or inhibiting further development of the disease, condition, or disorder. Subjects in need of treatment include patients already suffering from the disease or disorder as well as those prone to have the disorder or those in whom the disorder is to be prevented. As used herein, the terms “treating,” or similar terminology, do not imply a cure for a malignancy or any other disease or condition; rather, this terminology is used to refer to any clinically detectable improvement in the disease, disorder, or condition being treated or alleviated, including, but not limited to, in the case of a malignancy, reduction of tumor burden, reduction of tumor size, reduction of tumor spread, reduction of development of metastases, killing of tumor cells, arrest of growth or division of tumor cells, improved susceptibility of tumor cells to anti-neoplastic agents, reduction of pain, improvement in physical or mental functioning, improvement in subjective well-being experienced by the patient, or any other clinically detectable improvement. Analogous parameters can be used for determining effective treatment of conditions other than malignancies and are well known in the art. As used herein, the term “therapeutically effective quantity” refers to the amount of a therapeutic agent which is sufficient to treat the disease, disorder, or condition treatable by a method according to the present invention, as described above.

Methods according to the present invention can be used for treatment of a wide range of malignancies including, but not necessarily limited to: (A) breast cancer, including: (1) ductal carcinoma, including ductal carcinoma in situ (DCIS) (comedocarcinoma, cribriform, papillary, micropapillary), infiltrating ductal carcinoma (IDC), tubular carcinoma, mucinous (colloid) carcinoma, papillary carcinoma, metaplastic carcinoma, and inflammatory carcinoma; (2) lobular carcinoma, including lobular carcinoma in situ (LCIS) and invasive lobular carcinoma; and (3) Paget's disease of the nipple; (B) cancers of the female reproductive system, including: (1) cancers of the cervix uteri, including cervical intraepithelial neoplasia (Grade I), cervical intraepithelial neoplasia (Grade II), cervical intraepithelial neoplasia (Grade III) (squamous cell carcinoma in situ), keratinizing squamous cell carcinoma, nonkeratinizing squamous cell carcinoma, verrucous carcinoma, adenocarcinoma in situ, adenocarcinoma in situ, endocervical type, endometrioid adenocarcinoma, clear cell adenocarcinoma, adenosquamous carcinoma, adenoid cystic carcinoma, small cell carcinoma, and undifferentiated carcinoma; (2) cancers of the corpus uteri, including endometrioid carcinoma, adenocarcinoma, adenocanthoma (adenocarcinoma with squamous metaplasia), adenosquamous carcinoma (mixed adenocarcinoma and squamous cell carcinoma, mucinous adenocarcinoma, serous adenocarcinoma, clear cell adenocarcinoma, squamous cell adenocarcinoma, and undifferentiated adenocarcinoma; (3) cancers of the ovary, including serous cystadenoma. serous cystadenocarcinoma, mucinous cystadenoma, mucinous cystadenocarcinoma, endometrioid tumor, endometrioid adenocarcinoma, clear cell tumor, clear cell cystadenocarcinoma, and unclassified tumor; (4) cancers of the vagina, including squamous cell carcinoma and adenocarcinoma; and (5) cancers of the vulva, including vulvar intraepithelial neoplasia (Grade I), vulvar intraepithelial neoplasia (Grade II), vulvar intraepithelial neoplasia (Grade III) (squamous cell carcinoma in situ); squamous cell carcinoma, verrucous carcinoma, Paget's disease of the vulva, adenocarcinoma (NOS), basal cell carcinoma (NOS), and Bartholin's gland carcinoma; (C) cancers of the male reproductive system, including: (1) cancers of the penis, including squamous cell carcinoma; (2) cancers of the prostate, including adenocarcinoma, sarcoma, and transitional cell carcinoma of the prostate; (3) cancers of the testis, including seminomatous tumor, nonseminomatous tumor, teratoma, embryonal carcinoma, yolk sac tumor, and Choriocarcinoma; (D) cancers of the cardiac system, including sarcoma (angiosarcoma, fibrosarcoma, rhabdomyosarcoma, liposarcoma), myxoma, rhabdomyoma, fibroma, lipoma and teratoma; (E) cancers of the respiratory system, including squamous cell carcinoma of the larynx, primary pleural mesothelioma, and squamous cell carcinoma of the pharynx; (F) cancers of the lung, including squamous cell carcinoma (epidermoid carcinoma), variants of squamous cell carcinoma, spindle cell carcinoma, small cell carcinoma, carcinoma of other cells, carcinoma of intermediate cell type, combined oat cell carcinoma, adenocarcinoma, acinar adenocarcinoma, papillary adenocarcinoma, bronchiolo-alveolar carcinoma, solid carcinoma with mucus formation, large cell carcinoma, giant cell carcinoma, clear cell carcinoma, and sarcoma; (G) cancers of the gastrointestinal tract, including: (1) cancers of the ampulla of Vater, including primary adenocarcinoma, carcinoid tumor, and lymphoma; (2) cancers of the anal canal, including adenocarcinoma, squamous cell carcinoma, and melanoma; (3) cancers of the extrahepatic bile ducts, including carcinoma in situ, adenocarcinoma, papillary adenocarcinoma, adenocarcinoma, intestinal type, mucinous adenocarcinoma, clear cell adenocarcinom, segnet-ring cell carcinoma, adenosquamous carcinoma, squamous cell carcinoma, small cell (oat) carcinoma, undifferentiated carcinoma, carcinoma (NOS), sarcoma, and carcinoid tumor; (4) cancers of the colon and rectum, including adenocarcinoma in situ, adenocarcinoma, mucinous adenocarcinoma (colloid type; greater than 50% mucinous carcinoma), signet ring cell carcinoma (greater than 50% signet ring cell), squamous cell (epidermoid) carcinoma, adenosquamous carcinoma, small cell (oat cell) carcinoma, undifferentiated carcinoma, carcinoma (NOS), sarcoma, lymphoma, and carcinoid tumor; (5) cancers of the esophagus, including squamous cell carcinoma, adenocarcinoma, leiomyosarcoma, and lymphoma; (6) cancers of the gallbladder, including adenocarcinoma, adenocarcinoma, intestinal type, adenosquamous carcinoma, carcinoma in situ, carcinoma (NOS), clear cell adenocarcinoma, mucinous adenocarcinoma, papillary adenocarcinoma, signet-ring cell carcinoma, small cell (oat cell) carcinoma, squamous cell carcinoma, and undifferentiated carcinoma; (7) cancers of the lip and oral cavity, including squamous cell carcinoma; (8) cancers of the liver, including hepatoma (hepatocellular carcinoma), cholangiocarcinoma, hepatoblastoma, angiosarcoma, hepatocellular adenoma, and hemangioma; (9) cancers of the exocrine pancreas, including duct cell carcinoma, pleomorphic giant cell carcinoma, giant cell carcinoma, osteoclastoid type, adenocarcinoma, adenosquamous carcinoma, mucinous (colloid) carcinoma, cystadenocarcinoma, acinar cell carcinoma, papillary carcinoma, small cell (oat cell) carcinoma, mixed cell typed, carcinoma (NOS), undifferentiated carcinoma, endocrine cell tumors arising in the islets of langerhans, and carcinoid; (10) cancers of the salivary glands, including acinic (acinar) cell carcinoma, adenoid cystic carcinoma (cylindroma), adenocarcinoma, squamous cell carcinoma, carcinoma in pleomorphic adenoma (malignant mixed tumor), mucoepidermoid carcinoma (well differentiated or low grade), and mucoepidermoid carcinoma (poorly differentiated or high grade); (11) cancers of the stomach, including adenocarcinoma, papillary adenocarcinoma, tubular adenocarcinoma, mucinous adenocarcinoma, signet ring cell carcinoma, adenosquamous carcinoma, squamous cell carcinoma, small cell carcinoma, undifferentiated carcinoma, lymphoma, sarcoma, and carcinoid tumor; and (12) cancers of the small intestine, including adenocarcinoma, lymphoma, carcinoid tumors, Kaposi's sarcoma, leiomyoma, hemangioma, lipoma, neurofibroma, and fibroma; (H) cancers of the urinary system, including: (1) cancers of the kidney, including renal cell carcinoma, carcinoma of Bellini's collecting ducts, adenocarcinoma, papillary carcinoma, tubular carcinoma, granular cell carcinoma, clear cell carcinoma (hypernephroma), sarcoma of the kidney, and nephroblastoma; (2) cancers of the renal pelvis and ureter, including transitional cell carcinoma, papillary transitional cell carcinoma, squamous cell carcinoma, and adenocarcinoma; (3) cancers of the urethra, including transitional cell carcinoma, squamous cell carcinoma, and adenocarcinoma; and (4) cancers of the urinary bladder, including carcinoma in situ, transitional urothelial cell carcinoma, papillary transitional cell carcinoma, squamous cell carcinoma, adenocarcinoma, undifferentiated; (I) cancers of muscle, bone, and soft tissue, including: (1) cancers of bone, including: (a) bone-forming: osteosarcoma; (b) cartilage-forming: chondrosarcoma and mesenchymal chondrosarcoma; (c) diant cell tumor, malignant; (d) Ewing's sarcoma; (e) vascular tumors: hemangioendothelioma, hemangiopericytoma, and angiosarcoma; (f) connective tissue tumors: fibrosarcoma, liposarcoma, malignant mesenchymoma, and undifferentiated sarcoma; and (g) other tumors: chordoma and adamantinoma of long bones; (2) cancers of soft tissues, including: alveolar soft-part sarcoma, angiosarcoma, epithelioid sarcoma, extraskeletal chondrosarcoma, fibrosarcoma, leiomyosarcoma, liposarcoma, malignant fibrous histiocytoma, malignant hemangiopericytoma, malignant mesenchymoma, malignant schwannoma, rhabdomyosarcoma, synovial sarcoma, and sarcoma (NOS); (3) cancers of the nervous system, including cancers of the skull (osteoma, hemangioma, granuloma, xanthoma, osteitis deformans), cancers of the meninges (meningioma, meningiosarcoma, gliomatosis), cancers of the brain (astrocytoma, medulloblastoma, glioma, ependymoma, germinoma (pilealoma), glioblastoma multiform, oligodendroglioma, schwannoma, retinoblastoma, congenital tumors), and cancers of the spinal cord neurofibroma, meningioma, glioma, sarcoma); (4) hematologic cancers, including myeloid leukemia (acute and chronic), acute lymphloblastic leukemia, chronic lymphocytic leukemia, myeloproliferative diseases, multiple myeloma; myelodysplastic syndrome), Hodgkin's disease, and non-Hodgkin's lymphoma (malignant lymphoma); (5) cancers of the endocrine system, including: (a) cancers of the thyroid gland, including papillary carcinoma (including those with follicular foci), follicular carcinoma, medullary carcinoma, and undifferentiated (anaplastic) carcinoma; and (b) neuroblastomas, including sympathicoblastoma, sympathicogonioma, malignant ganglioneuroma, gangliosympathicoblastoma, and ganglioneuroma; (6) cancers of the skin, including squamous cell carcinoma, spindle cell variant of squamous cell carcinoma, basal cell carcinoma, adenocarcinoma developing from sweat or sebaceous gland, and malignant melanoma; (7) cancers of the eye, including: (a) cancers of the conjunctiva, including carcinoma of the conjunctiva; (b) cancers of the eyelid, including basal cell carcinoma, squamous cell carcinoma, melanoma of the eyelid, and sebaceous cell carcinoma; (c) cancers of the lacrimal gland, including adenocarcinoma, adenoid cystic carcinoma, carcinoma in pleomorphic adenoma, mucoepidermoid carcinoma, and squamous cell carcinoma; (d) cancers of the uvea, including spindle cell melanoma, mixed cell melanoma, and epithelioid cell melanoma; (e) cancers of the orbit, including sarcoma of the orbit, soft tissue tumor, and sarcoma of bone; and (f) retinoblastoma. As detailed above, methods according to the present invention are particularly applicable to prostate cancer.

The additional therapeutic agents described above can be administered directly to subjects in need of treatment. However, additional therapeutic agents are preferably administered to the subjects in pharmaceutical compositions which comprise the therapeutic agent, and, optionally, other therapeutically active agents in a therapeutically effective dose along with a pharmaceutically acceptable carrier, diluent or excipient in unit dosage form. Pharmaceutically acceptable carriers are agents which are not biologically or otherwise undesirable, i.e., the agents can be administered to a subject along with the therapeutic agent without causing any undesirable biological effects or interacting in a deleterious manner with any of the components of the pharmaceutical composition in which it is contained. The compositions can additionally contain other therapeutic agents, as described above, that are suitable for treating or preventing the disease, disorder, or condition treatable by the therapeutic agent as described above, particularly malignancies. Pharmaceutically acceptable carriers enhance or stabilize the composition, or can facilitate preparation of the composition. Pharmaceutically acceptable carriers include solvents, dispersion media, coatings, antibacterial and antifungal agents, isotonic and absorption delaying agents, and the like that are physiologically compatible. The pharmaceutically acceptable carrier should be suitable for various routes of administration described herein.

A pharmaceutical composition containing a therapeutic composition incorporating a therapeutic agent and/or other therapeutic agents can be administered by a variety of methods known in the art. The routes and/or modes of administration vary depending upon the desired results. Depending on the route of administration, the therapeutic agent may be coated in a material to protect the therapeutic agent from the action of acids and other compounds that may inactivate the agent. Conventional pharmaceutical practice can be employed to provide suitable formulations or compositions for the administration of such pharmaceutical compositions to subjects. Any appropriate route of administration can be employed, for example, but not limited to, intravenous, parenteral, intraperitoneal, intravenous, transcutaneous, subcutaneous, intramuscular, intraurethral, or oral administration. Depending on the severity of the malignancy or other disease, disorder, or condition to be treated, as well as other conditions affecting of the subject to be treated, either systemic or localized delivery of the pharmaceutical composition can be used in the course of treatment. The pharmaceutical composition as described above can be administered together with additional therapeutic agents intended to treat a particular disease or condition, which may be the same disease or condition that the therapeutic agent incorporated in the pharmaceutical composition is intended to treat, which may be a related disease or condition, or which even may be an unrelated disease or condition. The additional therapeutic agent or agents can be administered simultaneously or at different times.

In some embodiments, local administration of the pharmaceutical composition is desired in order to achieve the intended therapeutic effect. Many methods of localized delivery of therapeutic agents can be used in the practice of the invention. For example, a therapeutic agent can be administered directly to the site of the malignancy by direct injection or infusion or by other means known in the art.

Pharmaceutical compositions as described above can be prepared in accordance with methods well known and routinely practiced in the art. See, e.g., Remington: The Science and Practice of Pharmacy, Mack Publishing Co., 20^{th} ed., 2000; and Sustained and Controlled Release Drug Delivery Systems, J. R. Robinson, ed., Marcel Dekker, Inc., New York, 1978. Pharmaceutical compositions are preferably manufactured under GMP conditions. Formulations for parenteral administration may, for example, contain excipients, sterile water, or saline, polyalkylene glycols such as polyethylene glycol, oils of vegetable origin, or hydrogenated naphthalenes. Biocompatible, biodegradable lactide polymers, lactide/glycolide copolymers, or polyoxyethylene-polyoxypropylene copolymers may be used to control the release of the compounds. Other potentially useful parenteral delivery systems for molecules of the invention include ethylene-vinyl acetate copolymer particles, osmotic pumps, and implantable infusion systems. Formulations for inhalation may contain excipients, for example, lactose, or may be aqueous solutions containing, e.g., polyoxyethylene-9-lauryl ether, glycocholate and deoxycholate, or can be oily solutions for administration or gels.

Actual dosage levels of the active ingredients in the pharmaceutical compositions can be varied so as to obtain an amount of the active ingredient which is effective to achieve the desired therapeutic response for a particular subject, composition, and mode of administration, without being toxic to the subject. The selected dosage level depends upon a variety of pharmacokinetic factors including the activity of the particular therapeutic agent, the route of administration, the time of administration, the rate of excretion of the particular compound being employed, the severity of the condition, other health considerations affecting the subject, and the status of liver and kidney function of the subject. It also depends on the duration of the treatment, other drugs, compounds and/or materials used in combination with the particular therapeutic agent employed, as well as the age, weight, condition, general health and prior medical history of the subject being treated, and like factors. Methods for determining optimal dosages are described in the art, e.g., Remington: The Science and Practice of Pharmacy, Mack Publishing Co., 20^{th} ed., 2000. Typically, a pharmaceutically effective dosage would be between about 0.001 and 100 mg/kg body weight of the subject to be treated. Similar considerations apply if additional therapeutic agents are administered as described above.

The therapeutic agent, and, if desired, other therapeutic agents described above, are usually administered to the subjects on multiple occasions. Intervals between single dosages can be weekly, monthly or yearly. Intervals can also be irregular as indicated by therapeutic response or other parameters well known in the art. Alternatively, the therapeutic agent can be administered as a sustained release formulation, in which case less frequent administration is required. Dosage and frequency vary depending on the half-life in the subject of the therapeutic agent and the other drugs, if any, included in a pharmaceutical composition.

For the purposes of the present application, treatment can be monitored by observing one or more of the improving symptoms associated with the disease, disorder, or condition being treated, or by observing one or more of the improving clinical parameters associated with the disease, disorder, or condition being treated, as described above.

Preparations formulated for oral administration may be in the form of tablets, dragees, capsules, or solutions. The pharmaceutical compositions contemplated by the present invention may be manufactured in a manner that is itself known, e.g., by means of conventional mixing, dissolving, granulating, dragee-making, levitating, emulsifying, encapsulating, entrapping or lyophilizing processes.

Pharmaceutical formulations for parenteral administration can include aqueous solutions or suspensions. Suitable lipophilic solvents or vehicles include fatty oils such as sesame oil or synthetic fatty acid esters, such as ethyl oleate or triglycerides. Aqueous injection suspensions may contain substances which increase the viscosity of the suspension, such as sodium carboxymethyl cellulose, sorbitol, or dextran. Optionally, the suspension may also contain suitable stabilizers or modulators which increase the solubility or dispersibility of the liposome composition to allow for the preparation of highly concentrated solutions. Pharmaceutical preparations for oral use can be obtained by combining the liposome compositions with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating modulators may be added, such as the cross-linked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different doses of therapeutic agent.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the liposome composition may be dissolved or suspended in suitable liquids, such as fatty oils, liquid paraffin, or liquid polyethylene glycols. In addition, stabilizers may be added.

Other ingredients such as stabilizers, for example, antioxidants such as sodium citrate, ascorbyl palmitate, propyl gallate, reducing agents, ascorbic acid, vitamin E, sodium bisulfite, butylated hydroxytoluene, BHA, acetylcysteine, monothioglycerol, phenyl-α-naphthylamine, or lecithin can be used. Also, chelators such as EDTA can be used. Other ingredients that are conventional in the area of pharmaceutical compositions and formulations, such as lubricants in tablets or pills, coloring agents, or flavoring agents, can be used. Also, conventional pharmaceutical excipients or carriers can be used. The pharmaceutical excipients can include, but are not necessarily limited to, calcium carbonate, calcium phosphate, various sugars or types of starch, cellulose derivatives, gelatin, vegetable oils, polyethylene glycols and physiologically compatible solvents. Other pharmaceutical excipients are well known in the art. Exemplary pharmaceutically acceptable carriers include, but are not limited to, any and/or all of solvents, including aqueous and non-aqueous solvents, dispersion media, coatings, antibacterial and/or antifungal agents, isotonic and/or absorption delaying agents, and/or the like. The use of such media and/or agents for pharmaceutically active substances is well known in the art. Except insofar as any conventional medium, carrier, or agent is incompatible with the active ingredient or ingredients, its use in a composition according to the present invention is contemplated. Supplementary active ingredients can also be incorporated into the compositions, particularly as described above. For administration of any of the compounds used in the present invention, preparations should meet sterility, pyrogenicity, general safety, and purity standards as required by the FDA Office of Biologics Standards or by other regulatory organizations regulating drugs.

Sustained-release formulations or controlled-release formulations are well-known in the art. For example, the sustained-release or controlled-release formulation can be (1) an oral matrix sustained-release or controlled-release formulation; (2) an oral multilayered sustained-release or controlled-release tablet formulation; (3) an oral multiparticulate sustained-release or controlled-release formulation; (4) an oral osmotic sustained-release or controlled-release formulation; (5) an oral chewable sustained-release or controlled-release formulation; or (6) a dermal sustained-release or controlled-release patch formulation.

The pharmacokinetic principles of controlled drug delivery are described, for example, in B. M. Silber et al., “Pharmacokinetic/Pharmacodynamic Basis of Controlled Drug Delivery” in Controlled Drug Delivery: Fundamentals and Applications (J. R. Robinson & V. H. L. Lee, eds, 2d ed., Marcel Dekker, New York, 1987), ch. 5, pp. 213-251, incorporated herein by this reference.

One of ordinary skill in the art can readily prepare formulations for controlled release or sustained release comprising a therapeutic agent as described above by modifying the formulations described above, such as according to principles disclosed in V. H. K. Li et al, “Influence of Drug Properties and Routes of Drug Administration on the Design of Sustained and Controlled Release Systems” in Controlled Drug Delivery: Fundamentals and Applications (J. R. Robinson & V. H. L. Lee, eds, 2d ed., Marcel Dekker, New York, 1987), ch. 1, pp. 3-94, incorporated herein by this reference. This process of preparation typically takes into account physicochemical properties of the therapeutic compound, such as aqueous solubility, partition coefficient, molecular size, stability, and nonspecific binding to proteins and other biological macromolecules. This process of preparation also takes into account biological factors, such as absorption, distribution, metabolism, duration of action, the possible existence of side effects, and margin of safety, for the therapeutic composition. Accordingly, one of ordinary skill in the art could modify the formulations into a formulation having the desirable properties described above for a particular application.

Yet another aspect of the invention is a kit for use in treating malignancies. In general, this kit comprises, separately packaged:

(1) a culture of viable neural stem cells such that the neural stem cells can be transplanted to a post-surgical site of the malignancy; and

(2) a biocompatible adhesive as described above for adhering the neural stem cells to the post-surgical site of the malignancy.

The kit can further include one or more of the following, separately packaged:

(1) instructions for use;

(2) an applicator to apply the biocompatible adhesive to the post-surgical site of the malignancy;

(3) an applicator to apply the neural stem cells to the post-surgical site of the malignancy;

(4) at least one survival factor as described above; and

(4) at least one anti-neoplastic therapeutic agent as described above.

If the kit further includes an applicator or applicators, a single applicator can be included that is suitable for application of both the biocompatible adhesive and the neural stem cells. Alternatively, the kit can include: (i) an applicator for application of the biocompatible adhesive; (ii) an applicator for application of the neural stem cells; or (iii) two applicators, one for application of the biocompatible adhesive, and one for application of the neural stem cells.

The at least one survival factor that can be incorporated in the kit is one or more of the survival factors described above.

The kit can be adapted for the treatment of prostate cancer.

The at least one anti-neoplastic therapeutic agent that can be incorporated in the kit is one or more of the anti-neoplastic therapeutic agents described above. The anti-neoplastic therapeutic agent can be an anti-neoplastic therapeutic agent suitable for the treatment of prostate cancer if the kit is adapted for the treatment of prostate cancer.

The following references are cited by parenthetical number above. These references are incorporated herein in their entirety by this reference.
1. Gu, C. et al. Semaphorin 3E and plexin-D1 control vascular pattern independently of neuropilins. Science 307, 265-8 (2005).
2. Jeon, H. G. et al. Perineural invasion is a prognostic factor for biochemical failure after radical prostatectomy. Int J Urol 16, 682-6 (2009).
3. Liebig, C. et al. Perineural invasion is an independent predictor of outcome in colorectal cancer. J Clin Oncol 27, 5131-7 (2009).
4. Mitsunaga, S. et al. Detail histologic analysis of nerve plexus invasion in invasive ductal carcinoma of the pancreas and its prognostic impact. Am J Surg Pathol 31, 1636-44 (2007).
5. Andreu-Agullo, C., Morante-Redolat, J. M., Delgado, A. C. & Farinas, I. Vascular niche factor PEDF modulates Notch-dependent stemness in the adult subependymal zone. Nat Neurosci 12, 1514-23 (2009).
6. Ramirez-Castillejo, C. et al. Pigment epithelium-derived factor is a niche signal for neural stem cell renewal. Nat Neurosci 9, 331-9 (2006).
7. Androutsellis-Theotokis, A. et al. Notch signalling regulates stem cell numbers in vitro and in vivo. Nature 442, 823-6 (2006).
8. Dawson, D. W. et al. Pigment epithelium-derived factor: a potent inhibitor of angiogenesis. Science 285, 245-8 (1999).
9. Doll, J. A. et al. Pigment epithelium-derived factor regulates the vasculature and mass of the prostate and pancreas. Nat Med 9, 774-80 (2003).
10. Yan, M. et al. Chronic DLL4 blockade induces vascular neoplasms. Nature 463, E6-7 (2010).
11. Gridley, T. Notch signaling in vascular development and physiology. Development 134, 2709-18 (2007).
12. Kniel, P. C., Junker, U., Perrin, I. V., Bestetti, G. E. & Rossi, G. L. Varied effects of experimental diabetes on the autonomic nervous system of the rat. Lab Invest 54, 523-30 (1986).
13. Timperley, W. R., Boulton, A. J., Davies-Jones, G. A., Jarratt, J. A. & Ward, J. D. Small vessel disease in progressive diabetic neuropathy associated with good metabolic control. J Clin Pathol 38, 1030-8 (1985).
14. Ceyhan, G. O. et al. Pancreatic neuropathy results in “neural remodeling” and altered pancreatic innervation in chronic pancreatitis and pancreatic cancer. Am J Gastroenterol 104, 2555-65 (2009).
15. Ashraf, S. et al. The absence of autonomic perivascular nerves in human colorectal liver metastases. Br J Cancer 73, 349-59 (1996).
16. Mitchell, B. S., Schumacher, U. & Kaiserling, E. Are tumours innervated? Immunohistological investigations using antibodies against the neuronal marker protein gene product 9.5 (PGP 9.5) in benign, malignant and experimental tumours. Tumour Biol 15, 269-74 (1994).
17. Powell, M. S. et al. Neuroanatomy of the normal prostate. Prostate 65, 52-7 (2005).
18. Caygill, C. P., Knowles, R. L. & Hall, R. Increased risk of cancer mortality after vagotomy for peptic ulcer: a preliminary analysis. Eur J Cancer Prey 1, 35-7 (1991).
19. Erin, N., Akdas Barkan, G., Harms, J. F. & Clawson, G. A. Vagotomy enhances experimental metastases of 4THMpc breast cancer cells and alters substance P level. Regul Pept 151, 35-42 (2008).
20. Ayala, G. E. et al. Cancer-related axonogenesis and neurogenesis in prostate cancer. Clin Cancer Res 14, 7593-603 (2008).
21. Liebig, C., Ayala, G., Wilks, J. A., Berger, D. H. & Albo, D. Perineural invasion in cancer: a review of the literature. Cancer 115, 3379-91 (2009).
22. Woodhoo, A. et al. Notch controls embryonic Schwann cell differentiation, postnatal myelination and adult plasticity. Nat Neurosci 12, 839-47 (2009).
23. Imai, D., Yoneya, S., Gehlbach, P. L., Wei, L. L. & Mori, K. Intraocular gene transfer of pigment epithelium-derived factor rescues photoreceptors from light-induced cell death. J Cell Physiol 202, 570-8 (2005).
24. Murakami, Y. et al. Inhibition of nuclear translocation of apoptosis-inducing factor is an essential mechanism of the neuroprotective activity of pigment epithelium-derived factor in a rat model of retinal degeneration. Am J Pathol 173, 1326-38 (2008).
25. Gutierrez, J. C., Fischer, A. C., Sola, J. E., Perez, E. A. & Koniaris, L. G. Markedly improving survival of neuroblastoma: a 30-year analysis of 1,646 patients. Pediatr Surg Int 23, 637-46 (2007).
26. Mahadevan, D. & Von Hoff, D. D. Tumor-stroma interactions in pancreatic ductal adenocarcinoma. Mol Cancer Ther 6, 1186-97 (2007).
27. Calicchio, M. L., Collins, T. & Kozakewich, H. P. Identification of signaling systems in proliferating and involuting phase infantile hemangiomas by genome-wide transcriptional profiling. Am J Pathol 174, 1638-49 (2009).
28. Rosenstein, J. M. & Krum, J. M. New roles for VEGF in nervous tissue—beyond blood vessels. Exp Neurol 187, 246-53 (2004).
29. Zachary, I. Neuroprotective role of vascular endothelial growth factor: signalling mechanisms, biological function, and therapeutic potential. Neurosignals 14, 207-21 (2005).
30. Bull, N. D. & Bartlett, P. F. The adult mouse hippocampal progenitor is neurogenic but not a stem cell. J Neurosci 25, 10815-21 (2005).

### ADVANTAGES OF THE INVENTION

The present invention provides a new route and mechanism for treatment of malignancies that is based on the previously unexploited interaction between neural stem cells and angiogenesis of tumor tissue. The use of this interaction provides a new avenue for treatment of a wide range of malignancies. The results presented herein show that this route and mechanism are particularly suitable for the treatment of prostate cancer.

Methods according to the present invention are free of side effects and can be employed together with other conventional means for treatment of malignancies, such as surgery and radiation. These methods do not depend on specific interactions between markers on the tumor cells and any particular anti-neoplastic drug, which means that these methods are broadly applicable to a wide range of malignancies and do not depend for their applicability on specific tumor markers or specific histologic characteristics of malignancies.

Methods according to the present invention possess industrial applicability for the preparation of a medicament for the treatment of malignancies. Kits according to the present invention possess industrial applicability as articles of manufacture possessing a clearly defined use in the treatment of malignancies.

With respect to ranges of values, the invention encompasses each intervening value between the upper and lower limits of the range to at least a tenth of the lower limit's unit, unless the context clearly indicates otherwise. Moreover, the invention encompasses any other stated intervening values and ranges including either or both of the upper and lower limits of the range, unless specifically excluded from the stated range.

Unless defined otherwise, the meanings of all technical and scientific terms used herein are those commonly understood by one of ordinary skill in the art to which this invention belongs. One of ordinary skill in the art will also appreciate that any methods and materials similar or equivalent to those described herein can also be used to practice or test this invention.

The publications and patents discussed herein are provided solely for their disclosure prior to the filing date of the present application. Nothing herein is to be construed as an admission that the present invention is not entitled to antedate such publication by virtue of prior invention. Further the dates of publication provided may be different from the actual publication dates which may need to be independently confirmed.

All the publications cited are incorporated herein by reference in their entireties, including all published patents, patent applications, and literature references, as well as those publications that have been incorporated in those published documents. However, to the extent that any publication incorporated herein by reference refers to information to be published, applicants do not admit that any such information published after the filing date of this application to be prior art.

As used in this specification and in the appended claims, the singular forms include the plural forms. For example the terms “a,” “an,” and “the” include plural references unless the content clearly dictates otherwise. Additionally, the term “at least” preceding a series of elements is to be understood as referring to every element in the series. The inventions illustratively described herein can suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the future shown and described or any portion thereof, and it is recognized that various modifications are possible within the scope of the invention claimed. Thus, it should be understood that although the present invention has been specifically disclosed by preferred embodiments and optional features, modification and variation of the inventions herein disclosed can be resorted by those skilled in the art, and that such modifications and variations are considered to be within the scope of the inventions disclosed herein. The inventions have been described broadly and generically herein. Each of the narrower species and subgeneric groupings falling within the scope of the generic disclosure also form part of these inventions. This includes the generic description of each invention with a proviso or negative limitation removing any subject matter from the genus, regardless of whether or not the excised materials specifically resided therein. In addition, where features or aspects of an invention are described in terms of the Markush group, those schooled in the art will recognize that the invention is also thereby described in terms of any individual member or subgroup of members of the Markush group. It is also to be understood that the above description is intended to be illustrative and not restrictive. Many embodiments will be apparent to those of in the art upon reviewing the above description. The scope of the invention should therefore, be determined not with reference to the above description, but should instead be determined with reference to the appended claims, along with the full scope of equivalents to which such claims are entitled. Those skilled in the art will recognize, or will be able to ascertain using no more than routine experimentation, many equivalents to the specific embodiments of the invention described. Such equivalents are intended to be encompassed by the following claims.

I claim:

1. A method for treating a malignancy comprising the steps of:
(a) harvesting neural stem cells;
(b) culturing neural stem cells under conditions such that the cells proliferate while retaining their ability to differentiate;
(c) applying a biocompatible adhesive to a post-surgical site of a malignancy; and
(d) following the application of the biocompatible adhesive to the post-surgical site of the malignancy, applying the cultured neural stem cells to the post-surgical site of the malignancy to remove residual tumor cells and to induce endothelial cell apoptosis.

2. The method of claim 1 wherein the step of harvesting neural stem cells is performed by harvesting neural stem cells from bone marrow.

3. The method of claim 1 wherein the step of harvesting neural stem cells is performed by harvesting neural stem cells from adipose tissue.

4. The method of claim 1 wherein the step of harvesting neural stem cells is performed by harvesting neural stem cells derived from hematopoietic stem cells.

5. The method of claim 1 wherein the step of harvesting neural stem cells is performed by harvesting neural stem cells derived from Schwann cells from peripheral nerve biopsies.

6. The method of claim 1 wherein the step of harvesting the neural stem cells is performed by using a high efficiency cell sorter to retrieve the neural stem cells, wherein the high efficiency cell sorter sorts cells by fluorescence-activated cell sorting employing fluorochrome-conjugated antibodies, and wherein the fluorochrome-conjugated antibodies specifically bind a marker selected from the group consisting of nestin, PAX6, CD44, SOX2, RC2, BLBP, and GLAST.

7. The method of claim 1 wherein the step of harvesting the neural stem cells is performed by magnetic separation.

8. The method of claim 1 wherein the harvested neural stem cells are cultured.

9. The method of claim 8 wherein proliferation of the harvested neural stem cells is stimulated by treatment with at least one growth factor selected from the group consisting of bFGF (basic fibroblast growth factor), EGF, TGF-α, and aFGF.

10. The method of claim 9 wherein proliferation of the harvested neural stem cells is stimulated by treatment with bFGF.

11. The method of claim 8 wherein the culture medium is DMEM/F12 medium.

12. The method of claim 11 wherein the DMEM/F12 medium is supplemented with N2 (Invitrogen), B27 (Invitrogen), 2 μg/ml heparin, 100 U/ml penicillin, and 100 μg/ml streptomycin.

13. The method of claim 11 wherein the culture medium includes at least one growth-promoting additive selected from the group consisting of insulin, transferrin, sodium selenite, putrescine, and progesterone.

14. The method of claim 13 wherein the culture medium includes at least one additional growth-promoting factor selected from the group consisting of pituitary extract, antibiotics, and fetal calf serum.

15. The method of claim 1 wherein the neural stem cells are obtained from a subject to be treated.

16. The method of claim 1 wherein the biocompatible adhesive is a DOPA-substituted polyethylene glycol polymer.

17. The method of claim 16 wherein the DOPA-substituted polyethylene glycol polymer has the structure 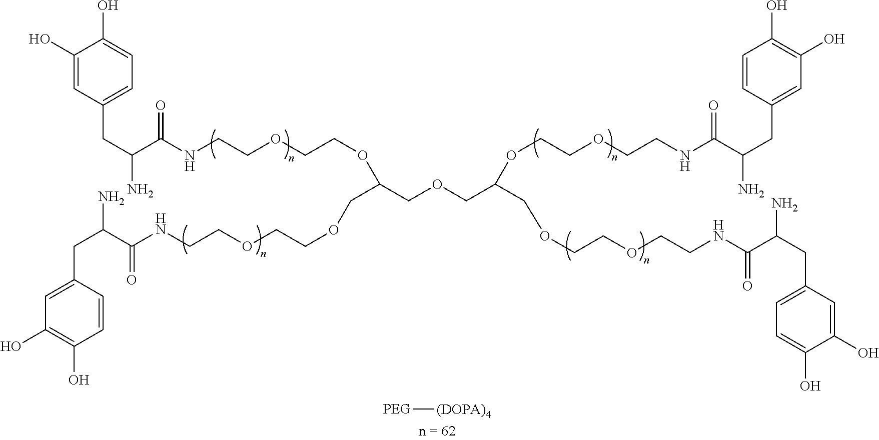
wherein n is from about 20 to about 100.

18. The method of claim 17 wherein n is from about 40 to about 80.

19. The method of claim 18 wherein n is 62.

20. The method of claim 1 wherein the biocompatible adhesive is selected from the group consisting of fibrin glues and alkyl-α-cyanoacrylate glues.

21. The method of claim 1 wherein the neural stem cells induce endothelial cell apoptosis.

22. The method of claim 1 wherein the neural stem cells release at least one anti-angiogenic protein.

23. The method of claim 22 wherein the at least one anti-angiogenic protein is selected from the group consisting of PEDF (pigment epithelium-derived factor), TSP-1 (thrombospondin-1), and angiostatin.

24. The method of claim 1 wherein the neural stem cells are applied to the post-operative site of the malignancy with at least one survival factor selected from the group consisting of pigment epithelium derived factor and propranolol.

25. The method of claim 1 wherein the method further comprises the step of administering a therapeutically effective quantity of an additional anti-neoplastic therapeutic agent.

26. The method of claim 25 wherein the additional anti-neoplastic therapeutic agent is selected from the group consisting of a nitrogen mustard, an alkylating agent, an alkyl sulfonate, a nitrosourea, a triazene, a folic acid analogue, a pyrimidine analogue antimetabolite, a purine analogue antimetabolite, a vinca alkaloid, a taxane, a camptothecin, an antibiotic, an enzyme, a biological response modifier, a platinum coordination complex, an anthracenedione, a substituted urea, a substituted hydrazine, an adrenocortical suppressant, a tyrosine kinase inhibitor, an adrenocorticosteroid, a progestin, an estrogen, an antiestrogen, an androgen, an antiandrogen, a gonadotropin-releasing hormone analogue, a monoclonal antibody, an interferon, an epidermal growth factor receptor (EGFR) kinase inhibitor, a vascular growth factor receptor (VGFR) kinase inhibitor, a fibroblast growth factor receptor (FGFR) kinase inhibitor, a platelet derived growth factor receptor (PDGF) kinase inhibitor, a Bcr-Ab1 kinase inhibitor, an antisense molecule, a non-steroidal anti-inflammatory drug, a topoisomerase inhibitor, a pro-apoptotic Bcl-2 family protein, a photodynamic compound, a radiodynamic compound, an immune suppressant, and an anti-angiogenic agent.

27. The method of claim 25 wherein the additional anti-neoplastic therapeutic agent is selected from the group consisting of mechlorethamine, cyclophosphamide, ifosfamide, melphalan, chlorambucil, hexamethylmelamine, thiotepa, busulfan, carmustine, streptozocin, dacarbazine, temozolomide, methotrexate, 5-fluorouracil, cytarabine, gemcitabine, 6-mercaptopurine, 6-thioguanine, pentostatin, vincristine, paclitaxel, docetaxel, topotecan, dactinomycin, daunorubicin, doxorubicin, bleomycin, mitomycin C, L-asparaginase, interferon-alfa, interleukin-2, cisplatin, carboplatin, mitoxantrone, hydroxyurea, N-methylhydrazine, mitotane, aminoglutethimide, imatinib, prednisone, prednisolone, methylprednisolone, dexamethasone, betamethasone, triamcinolone, hydroxyprogesterone caproate, medroxyprogesterone acetate, megestrol acetate, diethylstilbestrol, ethinyl estradiol, tamoxifen, anastrozole, testosterone propionate, fluoxymesterone, flutamine, leuprolide, trastuzumab, rituximab, alemtuzumab, bevacizumab, cetuximab, gemtuzumab, ibritumomab, panitumumab, tositumomab, raloxifene, bicalutamide, finasteride, ketoconazole, fludarabine, Mylotarg, etoposide, staurosprine, altretamine, capecitabine, cladribine, idarubicin, vinorelbine, geranylgeraniol, lomustine, semustine, hydroxymethylmelamine, plicamycin, azathioprine, 2-chlorodeoxyadenosine, 5-fluorodeoxyuridine, and batimistat.

28. The method of claim 1 wherein the method further comprises administration of a therapeutically effective quantity of anti-neoplastic ionizing radiation, wherein the anti-neoplastic ionizing radiation is administered by a method selected from the group consisting of X-ray administration, proton beam administration, brachytherapy, and radioisotope therapy, and where the anti-neoplastic ionizing radiation is administered by brachytherapy, the anti-neoplastic ionizing radiation is administered by brachytherapy with a radioisotope selected from the group consisting of cesium-137, cobalt-60, indium-192, iodine-125, palladium-103, and ruthenium-106.

29. The method of claim 28 wherein the radioisotope is conjugated to a targeting moiety selected from the group consisting of a hormone, a receptor target, and a monoclonal antibody.

30. The method of claim 28 wherein the anti-neoplastic ionizing radiation is administered by radiotherapy with a radioisotope selected from the group consisting of iodine-131, lutetium-177, yttrium-90, strontium-89, and samarium-153.

31. The method of claim 30 wherein the radioisotope is conjugated to a targeting moiety selected from the group consisting of a hormone, a receptor target, and a monoclonal antibody.

32. The method of claim 1 wherein the malignancy is prostate cancer.

33. A kit for use in treating malignancies comprising:
(a) a culture of viable neural stem cells such that the neural stem cells can be transplanted to a post-surgical site of the malignancy; and
(b) a biocompatible adhesive adhering the neural stem cells to the post-surgical site of the malignancy.

34. The kit of claim 33 wherein the biocompatible adhesive is a DOPA-substituted polyethylene glycol polymer.

35. The kit of claim 34 wherein the DOPA-substituted polyethylene glycol polymer has the structure 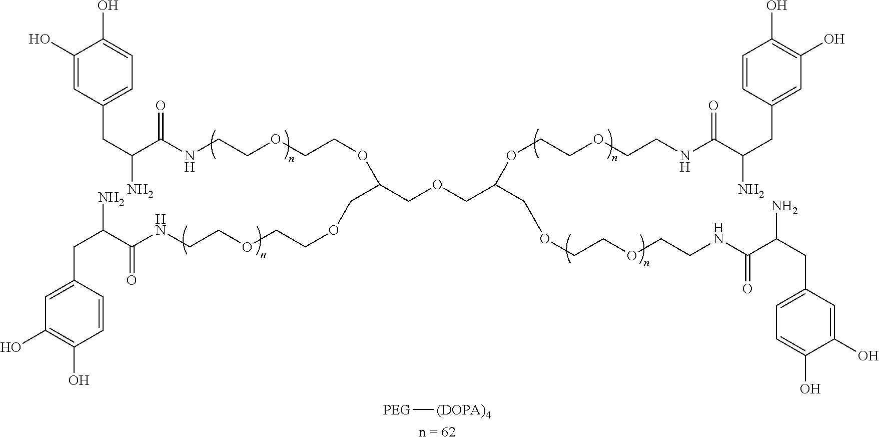
wherein n is from about 20 to about 100.

36. The kit of claim 35 wherein n is from about 40 to about 80.

37. The kit of claim 33 wherein the biocompatible adhesive is selected from the group consisting of fibrin glues and alkyl-α-cyanoacrylate glues.

38. The kit of claim 33 wherein the kit further comprises, separately packaged, at least one survival factor for the neural stem cells.

39. The kit of claim 38 wherein the at least one survival factor is selected from the group consisting of pigment epithelium derived factor (PEDF) and propranolol.

40. The kit of claim 33 wherein the kit further comprises, separately packaged, at least one anti-neoplastic therapeutic agent.

41. The kit of claim 40 wherein the at least one anti-neoplastic therapeutic agent is selected from the group consisting of a nitrogen mustard, an alkylating agent, an alkyl sulfonate, a nitrosourea, a triazene, a folic acid analogue, a pyrimidine analogue antimetabolite, a purine analogue antimetabolite, a vinca alkaloid, a taxane, a camptothecin, an antibiotic, an enzyme, a biological response modifier, a platinum coordination complex, an anthracenedione, a substituted urea, a substituted hydrazine, an adrenocortical suppressant, a tyrosine kinase inhibitor, an adrenocorticosteroid, a progestin, an estrogen, an antiestrogen, an androgen, an antiandrogen, a gonadotropin-releasing hormone analogue, a monoclonal antibody, an interferon, an epidermal growth factor receptor (EGFR) kinase inhibitor, a vascular growth factor receptor (VGFR) kinase inhibitor, a fibroblast growth factor receptor (FGFR) kinase inhibitor, a platelet derived growth factor receptor (PDGF) kinase inhibitor, a Bcr-Ab1 kinase inhibitor, an antisense molecule, a non-steroidal anti-inflammatory drug, a topoisomerase inhibitor, a pro-apoptotic Bcl-2 family protein, a photodynamic compound, a radiodynamic compound, an immune suppressant, and an anti-angiogenic agent.

42. The kit of claim 33 wherein the kit further comprises, separately packaged, an applicator to apply the biocompatible adhesive to the post-surgical site of the malignancy.

43. The kit of claim 33 wherein the kit further comprises, separately packaged, an applicator to apply the neural stem cells to the post-surgical site of the malignancy.

44. The kit of claim 33 wherein the kit further comprises, separately packaged, an applicator that is suitable for application of both the biocompatible adhesive and the neural stem cells to the post-surgical site of the malignancy.

45. The kit of claim 33 wherein the kit is adapted for the treatment of prostate cancer.
